(19) United States
(12) Patent Application Publication
(10) Pub. No.: US 2006/0270589 A1
(43) Pub. Date: Nov. 30, 2006

(54) Carotenoids, carotenoid analogs, or carotenoid derivatives for the treatment of visual disabilities

(76) Inventors: Samuel F. Lockwood, Lago Vista, TX (US); Geoff Nadolski, Kailua, HI (US)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/359,984

(22) Filed: Feb. 22, 2006

Related U.S. Application Data
(60) Provisional application No. 60/655,133, filed on Feb. 22, 2005.

(51) Int. Cl. A61K 38/16; A61K 31/7034; A61K 31/66; A61K 31/185; A61K 31/12; A61K 31/015

(52) U.S. Cl. 514/2; 514/690; 514/23; 514/125; 514/561; 514/763; 514/553

(57) ABSTRACT

A method and system used for treating visual disabilities using carotenoids, carotenoid analogs, and/or carotenoid derivatives. The analog, derivative, or intermediate may be administered such that a subject's risk of experiencing diseases associated with visual disabilities may be thereby reduced. Analogs or derivatives of carotenoids may include substituents including for example co-antioxidants (e.g., Vitamin C and Vitamin C analogs). The carotenoid analog or derivative may be synthetic. The carotenoid analog may include a conjugated polyene with between 7 to 14 double bonds. The conjugated polyene may include an acyclic alkene including at least one substituent and/or a cyclic ring including at least one substituent. In some embodiments, a carotenoid analog or derivative may include at least one substituent. The substituent may enhance the solubility of the carotenoid analog or derivative such that the carotenoid analog or derivative at least partially dissolves in water.

### PRIORITY CLAIM

This application claims priority to Provisional Patent Application No. 60/655,133 entitled “CAROTENOIDS, CAROTENOID ANALOGS, OR CAROTENOID DERIVATIVES FOR THE TREATMENT OF VISUAL DISABILITIES” filed on Feb. 22, 2005, which is incorporated by reference herein.

### BACKGROUND OF THE INVENTION

1. Field of the Invention

The invention generally relates to the fields of medicinal and synthetic chemistry. Specifically, the invention relates to the synthesis and use of carotenoids, including analogs, derivatives, and intermediates. More specifically, the invention relates to the use of carotenoids, carotenoid analogs or carotenoid derivatives for the inhibition and amelioration of diseases resulting in change of and/or loss of vision.

2. Description of the Relevant Art

Age-related macular degeneration (ARMD) is thought to be the result of a lifetime of oxidative insult that results in photoreceptor death within the macula. Increased risk of ARMD may result from low levels of lutein and zeaxanthin (macular pigment) in the diet, serum or retina, and excessive exposure to blue light. Through its light-screening capacity, antioxidant activity, and effects on chromatic aberration, macular pigment may reduce photooxidation in the central retina.

Nutritional treatment of retinal disease has proved at least partially successful in common retinitis pigmentosa (vitamin A), Bassen-Kowzweig disease (vitamins A, E, and K), gyrate atrophy (low protein, low arginine diet, and/or vitamin B6), Refsum disease (low phytol, low phytanic acid), and Sorsby fundus dystrophy (vitamin A). Small scale, prospective, double-masked, randomized, placebo-controlled studies have demonstrated that the progression of ARMD can be slowed with either zinc alone, or the combination of β-carotene, vitamin C, vitamin E, and zinc. The AREDS trial confirmed these findings. Studies have also evaluated the effect of lutein alone or lutein combined with additional carotenoids and antioxidants/minerals (including zinc, β-carotene, and vitamins C and E) on macular pigment optical density (MPOD) and objective visual outcome measures in atrophic ARMD. This study demonstrated that lutein alone or lutein combined with additional carotenoids and antioxidants/minerals (including zinc) significantly improved macular pigment optical density and glare recovery, improved near visual acuity, and significantly improved most measures of quality of vision (contrast sensitivity function), with lutein combined with antioxidants as well as a broad range of minerals having a broader effect. Lutein alone resulted in a net improvement in Amsler grid scotomas and metamorphopsia. These results are important because lutein is an essential carotenoid not produced by the body. Therapeutic loading and maintenance dosing of lutein and other nutrients may be required to treat atrophic ARMD. Due to absorptive saturation in the human GI tract, oral dosing of lutein and zeaxanthin supplements may not accomplish therapeutic treatment of these disease conditions; hence, parenteral dosing may be required utilizing pro- and/or soft-drugs.

Carotenoids are a group of natural pigments produced principally by plants, yeast, and microalgae. The family of related compounds now numbers greater than 700 described members, exclusive of Z and E isomers. Fifty (50) have been found in human sera or tissues. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranylgeranyl diphosphate molecules produces the parent C_{4b 0} carbon skeleton. Carotenoids without oxygenated functional groups are called “carotenes”, reflecting their hydrocarbon nature; oxygenated carotenes are known as “xanthophylls.” Cyclization at one or both ends of the molecule yields 7 identified end groups (illustrative structures shown in FIG. 1).

Documented carotenoid functions in nature include light-harvesting, photoprotection, and protective and sex-related coloration in microscopic organisms, mammals, and birds, respectively. A relatively recent observation has been the protective role of carotenoids against age-related diseases in humans as part of a complex antioxidant network within cells. This role is dictated by the close relationship between the physicochemical properties of individual carotenoids and their in vivo functions in organisms. The long system of alternating double and single bonds in the central part of the molecule (delocalizing the π-orbital electrons over the entire length of the polyene chain) confers the distinctive molecular shape, chemical reactivity, and light-absorbing properties of carotenoids. Additionally, isomerism around C═C double bonds yields distinctly different molecular structures that may be isolated as separate compounds (known as Z (“cis”) and E (“trans”), or geometric, isomers). Of the more than 700 described carotenoids, an even greater number of the theoretically possible mono-Z and poly-Z isomers are sometimes encountered in nature. The presence of a Z double bond creates greater steric hindrance between nearby hydrogen atoms and/or methyl groups, so that Z isomers are generally less stable thermodynamically, and more chemically reactive, than the corresponding all-E form. The all-E configuration is an extended, linear, and rigid molecule. Z-isomers are, by contrast, not simple, linear molecules (the so-called “bent-chain” isomers). The presence of any Z in the polyene chain creates a bent-chain molecule. The tendency of Z-isomers to crystallize or aggregate is much less than all-E, and Z isomers may sometimes be more readily solubilized, absorbed, and transported in vivo than their all-E counterparts. This has important implications for enteral (e.g., oral) and parenteral (e.g., intravenous, intra-arterial, intramuscular, intraperitoneal, intracoronary, and subcutaneous) dosing in mammals.

Carotenoids with chiral centers may exist either as the R (rectus) or S (sinister) configurations. As an example, astaxanthin (with 2 chiral centers at the 3 and 3′ carbons) may exist as 3 possible stereoisomers: 3S, 3′S; 3R, 3′S and 3S, 3′R (identical meso forms); or 3R, 3′R. The relative proportions of each of the stereoisomers may vary by natural source. For example, Haematococcus pluvialis microalgal meal is 99% 3S, 3′S astaxanthin, and is likely the predominant human evolutionary source of astaxanthin. Krill (3R, 3′R) and yeast sources yield different stereoisomer compositions than the microalgal source. Synthetic astaxanthin, produced by large manufacturers such as Hoffmann-LaRoche AG, Buckton Scott (USA), or BASF AG, are provided as defined geometric isomer mixtures of a 1:2:1 stereoisomer mixture (3S, 3′S; 3R, 3′S, (meso); 3R, 3′R) of non-esterified, free astaxanthin. Natural source astaxanthin from salmonid fish is predominantly a single stereoisomer (3S, 3′S), but does contain a mixture of geometric isomers. Astaxanthin from the natural source Haematococcuspluvialis may contain nearly 50% Z isomers. Similarly, lutein has 3 chiral centers, and may exist as 8 potential stereoisomers. The RRR-configuration predominates in the natural plant source material in the human diet, and is the only isomer found to any significant degree in the human retina. As stated above, the Z conformational change may lead to a higher steric interference between the two parts of the carotenoid molecule, rendering it less stable, more reactive, and more susceptible to reactivity at low oxygen tensions. In such a situation, in relation to the all-E form, the Z forms: (1) may be degraded first; (2) may better suppress the attack of cells by reactive oxygen species such as superoxide anion; and (3) may preferentially slow the formation of radicals. Overall, the Z forms may initially be thermodynamically favored to protect the lipophilic portions of the cell and the cell membrane from destruction. It is important to note, however, that the all-E form of astaxanthin, unlike β-carotene, retains significant oral bioavailability as well as antioxidant capacity in the form of its dihydroxy- and diketo-substitutions on the β-ionone rings, and has been demonstrated to have increased efficacy over β-carotene in most studies. The all-E form of astaxanthin has also been postulated to have the most membrane-stabilizing effect on cells in vivo. Therefore, it is likely that the all-E form of astaxanthin in natural and synthetic mixtures of stereoisomers is also extremely important in antioxidant mechanisms, and may be the form most suitable for particular pharmaceutical preparations.

The antioxidant mechanism(s) of carotenoids, and in particular astaxanthin, includes singlet oxygen quenching, direct radical scavenging, and lipid peroxidation chain-breaking. The polyene chain of the carotenoid absorbs the excited energy of singlet oxygen, effectively stabilizing the energy transfer by delocalization along the chain, and dissipates the energy to the local environment as heat. Transfer of energy from triplet-state chlorophyll (in plants) or other porphyrins and proto-porphyrins (in mammals) to carotenoids occurs much more readily than the alternative energy transfer to oxygen to form the highly reactive and destructive singlet oxygen (¹O₂). Carotenoids may also accept the excitation energy from singlet oxygen if any should be formed in situ, and again dissipate the energy as heat to the local environment. This singlet oxygen quenching ability has significant implications in cardiac ischemia, macular degeneration, porphyria, and other disease states in which production of singlet oxygen has damaging effects. In the physical quenching mechanism, the carotenoid molecule may be regenerated (most frequently), or be lost. Carotenoids are also excellent chain-breaking antioxidants, a mechanism important in inhibiting the peroxidation of lipids. Astaxanthin can donate a hydrogen (H) to the unstable polyunsaturated fatty acid (PUFA) radical, stopping the chain reaction. Peroxyl radicals may also, by addition to the polyene chain of carotenoids, be the proximate cause for lipid peroxide chain termination. The appropriate dose of astaxanthin has been shown to completely suppress the peroxyl radical chain reaction in liposome systems. Astaxanthin shares with vitamin E this dual antioxidant defense system of singlet oxygen quenching and direct radical scavenging, and in most instances (and particularly at low oxygen tension in vivo) is superior to vitamin E as a radical scavenger and physical quencher of singlet oxygen.

Carotenoids, and in particular astaxanthin, lutein, and zeaxanthin, are potent direct radical scavengers and singlet oxygen quenchers and possess all the desirable qualities of such therapeutic agents for inhibition or amelioration of ischemia-reperfusion injury. Synthesis of novel carotenoid derivatives with “soft-drug” properties (i.e. active as antioxidants in the derivatized form), with physiologically relevant, cleavable linkages to pro-moieties (e.g., phosphates and/or phosphate-linked soft drugs), can generate significant levels of free carotenoids in both plasma and solid organs. This quality alone may overcome difficulties with oral absorption in mammals, owing to the ability to deliver the compound parenterally. In the case of non-esterified, free astaxanthin, this is a particularly useful embodiment (characteristics specific to non-esterified, free astaxanthin below):
Lipid soluble in natural form; may be modified to become more water soluble;
Molecular weight of 597 Daltons (size<600 daltons (Da) readily crosses the blood brain barrier, or BBB);
Long polyene chain characteristic of carotenoids effective in singlet oxygen quenching and lipid peroxidation chain breaking; and
No pro-vitamin A activity in mammals (eliminating concerns of hypervitaminosis A and retinoid toxicity in humans).

The administration of antioxidants which are potent singlet oxygen quenchers and direct radical scavengers, particularly of superoxide anion, should limit hepatic fibrosis and the progression to cirrhosis by affecting the activation of hepatic stellate cells early in the fibrogenetic pathway. Reduction in the level of ROS by the administration of a potent antioxidant can therefore be crucial in the prevention of the activation of both HSC and Kupffer cells. This protective antioxidant effect appears to be spread across the range of potential therapeutic antioxidants, including water-soluble (e.g., vitamin C, glutathione, resveratrol) and lipophilic (e.g., vitamin E, β-carotene, astaxanthin) agents. Therefore, a co-antioxidant derivative strategy in which water-soluble and lipophilic agents are combined synthetically is a particularly useful embodiment. Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” to Lockwood et al. published on Jan. 13, 2005, as Publication No. US-2005-0009758 and U.S. patent application Ser. No. 11/106,378 filed on Apr. 14, 2005, entitled “CAROTENOID ANALOGS OR DERIVATIVES FOR THE INHIBITION AND AMELIORATION OF INFLAMMATION” to Lockwood et al. published on Nov. 24, 2005, as Publication No. U.S.-2005-0261254 both of which are incorporated by reference as if fully set forth herein.

Vitamin E is generally considered the reference antioxidant. When compared with vitamin E, carotenoids are more efficient in quenching singlet oxygen in homogenenous organic solvents and in liposome systems. They are better chain-breaking antioxidants as well in liposomal systems. They have demonstrated increased efficacy and potency in vivo. They are particularly effective at low oxygen tension, and in low concentration, making them extremely effective agents in disease conditions in which ischemia is an important part of the tissue injury and pathology. They are also effective in high light conditions in which the oxygen consumption is high, as, for example, in the retina. Lutein- and zeaxanthin-generating derivatives and/or analogs may provide excellent serum levels of free carotenoid, which can then be concentrated in the retina by normal physiologic mechanisms in vertebrates. These carotenoids also have a natural tropism for the heart and liver after oral administration. Therefore, therapeutic administration of carotenoids should provide a greater benefit in limiting fibrosis than vitamin E.

Problems related to the use of some carotenoids and structural carotenoid analogs or derivatives include: (1) the complex isomeric mixtures, including non-carotenoid contaminants, provided in natural and synthetic sources leading to costly increases in safety and efficacy tests required by such agencies as the FDA; (2) limited bioavailability upon administration to a subject; and (3) the differential induction of cytochrome P450 enzymes (this family of enzymes exhibits species-specific differences which must be taken into account when extrapolating animal work to human studies). Selection of the appropriate analog or derivative and isomer composition for a particular application increases the utility of carotenoid analogs or derivatives for the uses defined herein.

New methods and systems capable of stabilizing and/or improving visual acuity would be beneficial. Visual acuity may be stabilized and/or increased through methods and systems capable of macular repigmentation. Methods and systems capable of improving visual/optical parameters would be beneficial. Systems and methods capable of macular repigmentation would be beneficial in stabilizing/improving visual acuity and/or ARMD. Carotenoid analogs or derivatives displaying increased water-dispersibility would be beneficial for several reasons.

### SUMMARY

In some embodiments, a method of treating a visual disability in a subject may include administering to the subject an effective amount of a pharmaceutically acceptable composition or formulation comprising an analog or derivative of a carotenoid. The analog or derivative may be synthetic. The administration of analogs or derivatives of carotenoids may inhibit and/or ameliorate the occurrence of diseases in subjects which may affect visual acuity. In some embodiments, analogs or derivatives of carotenoids may be water-soluble.

In some embodiments, the administration of analogs or derivatives of carotenoids by one skilled in the art—including consideration of the pharmacokinetics and pharmacodynamics of therapeutic drug delivery—is expected to inhibit and/or ameliorate disease conditions associated with inhibition of vision.

“Water soluble” structural carotenoid analogs or derivatives are those analogs or derivatives which may be formulated in aqueous solution, either alone or with excipients. Water soluble carotenoid analogs or derivatives may include those compounds and synthetic derivatives which form molecular self-assemblies, and may be more properly termed “water dispersible” carotenoid analogs or derivatives. Water soluble and/or “water-dispersible” carotenoid analogs or derivatives may be preferred in some embodiments of the current invention.

Water soluble carotenoid analogs or derivatives may have a water solubility of greater than about 1 mg/mL in some embodiments. In certain embodiments, water soluble carotenoid analogs or derivatives may have a water solubility of greater than about 10 mg/mL. In some embodiments, water soluble carotenoid analogs or derivatives may have a water solubility of greater than about 50 mg/mL.

In some embodiments, water soluble analogs or derivatives of carotenoids may be administered to a subject alone or in combination with other carotenoid analogs or derivatives.

Embodiments may be further directed to pharmaceutical compositions comprising combinations of structural carotenoid analogs or derivatives to said subjects. The composition of an injectable structural carotenoid analog or derivative of lutein may be particularly useful in the therapeutic methods described herein. In yet a further embodiment, an injectable lutein structural analog or derivative is administered with a zeaxanthin structural analog or derivative and/or other carotenoid structural analogs or derivatives, or in formulation with antioxidants and/or excipients that further the intended purpose. In some embodiments, one or more of the lutein structural analogs or derivatives are water soluble.

As used herein, terms such as carotenoid analog and carotenoid derivative may generally refer to in some embodiments chemical compounds or compositions derived from a naturally occurring carotenoid. In some embodiments, terms such as carotenoid analog and carotenoid derivative may generally refer to chemical compounds or compositions which are synthetically derived from non-carotenoid based parent compounds; however, which ultimately substantially resemble a carotenoid derived analog. In certain embodiments, terms such as carotenoid analog and carotenoid derivative may generally refer to a synthetic derivative of a naturally occurring carotenoid. In some embodiments, a synthetic derivative of a naturally occurring carotenoid may not exist naturally (as is generally defined by what is generally known to one skilled in the art).

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 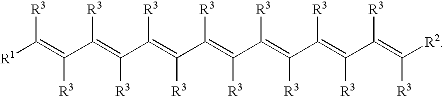
Each R³ may be independently hydrogen or methyl. Each R¹ and R² may be independently: 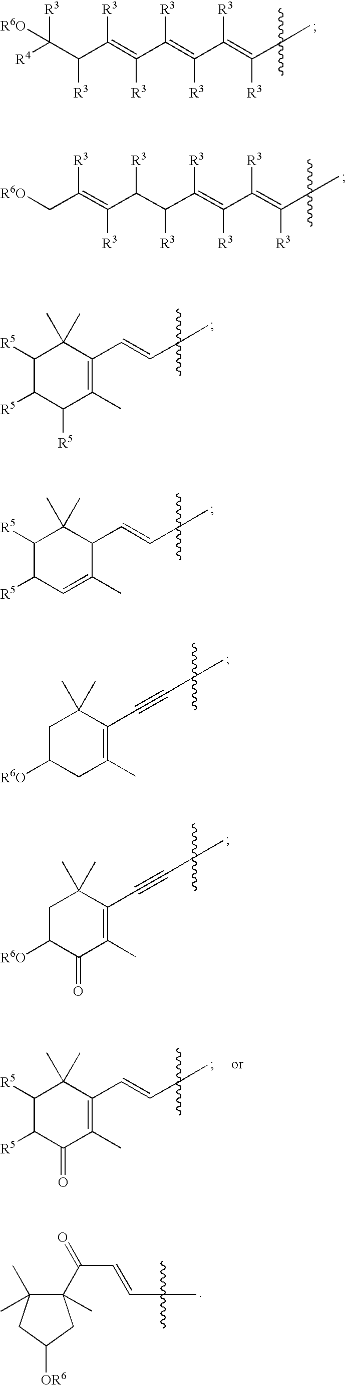
Each R⁴ may be independently hydrogen or methyl. Each R⁵ may be independently hydrogen, —OH, or —OR⁶. At least one R⁵ group may be —OR⁶. Each R⁶ may be independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant. Each R⁷ may be independently hydrogen, alkyl, or aryl. Each R⁸ may be hydrogen, alkyl, aryl, benzyl or a co-antioxidant. Each R⁹ may be hydrogen; alkyl; aryl; —P(O)(OR⁸)₂; —S(O)(OR⁸)₂; an amino acid; a peptide, a carbohydrate; a nucleoside, or a co-antioxidant. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 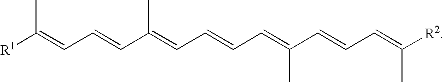
Each R¹ and R² may be independently: 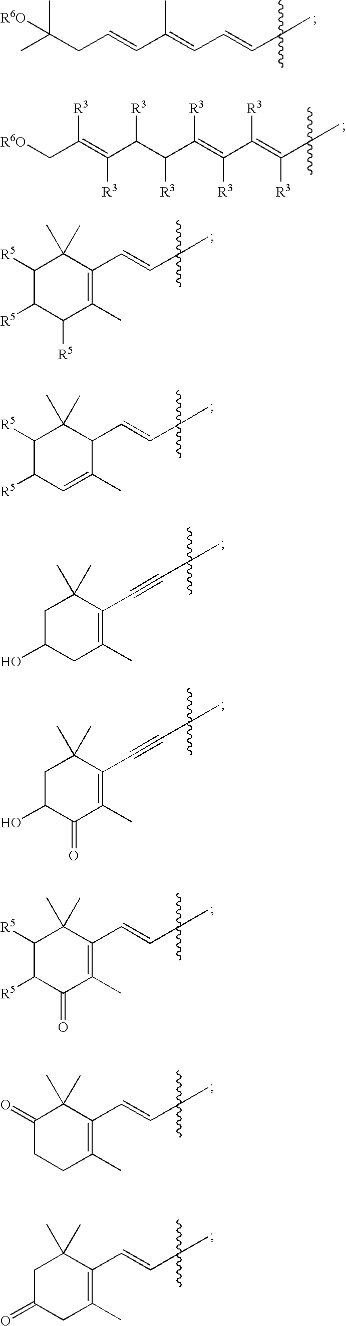 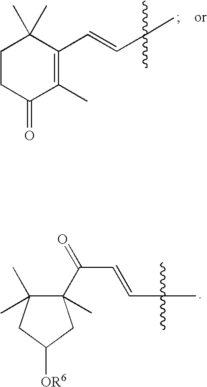
Each R⁴ may be independently hydrogen or methyl. Each R⁵ may be independently hydrogen, —OH, or —OR⁶. At least one R⁵ group may be —OR⁶. Each R⁶ may be independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂; -aryl-CO₂; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant. Each R⁷ may be independently hydrogen, alkyl, or aryl. Each R⁸ may be hydrogen, alkyl, aryl, benzyl or a co-antioxidant. Each R⁹ may be hydrogen; alkyl; aryl; —P(O)(OR⁸)₂; —S(O)(OR⁸)₂; an amino acid; a peptide, a carbohydrate; a nucleoside, or a co-antioxidant. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 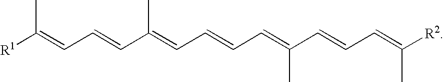
Each R¹ and R² may be independently: 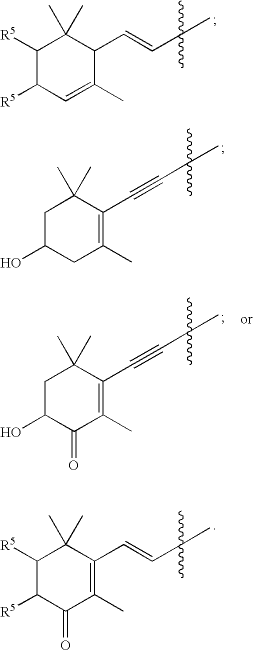
Each R⁵ may be independently hydrogen, —OH, or —OR⁶. At least one R⁵ group may be —OR⁶. Each R⁶ may be independently: 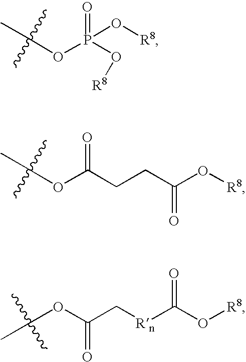
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 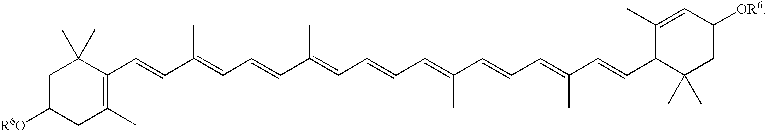
Each —OR⁶ may be independently: 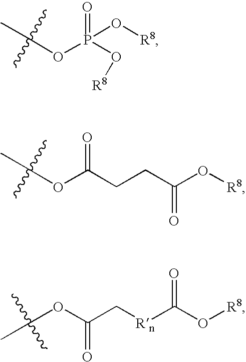
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 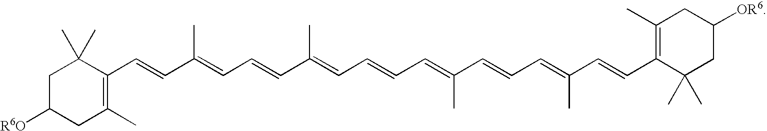
Each —OR⁶ may be independently: 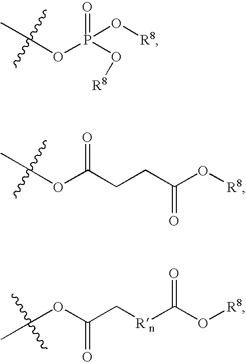
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including two or more carotenoid derivatives or analogs. The analog or derivative of the carotenoid may have the structures 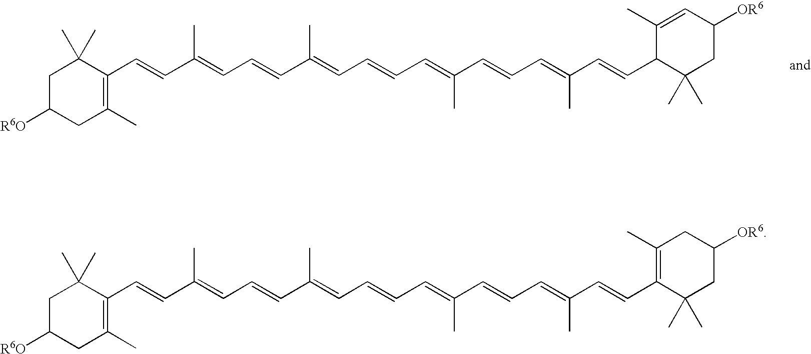
Each —OR⁶ may be independently: 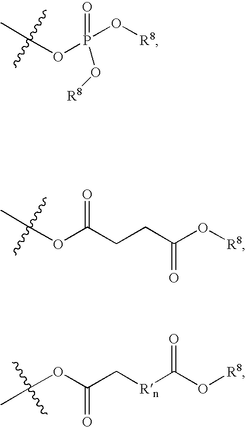
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 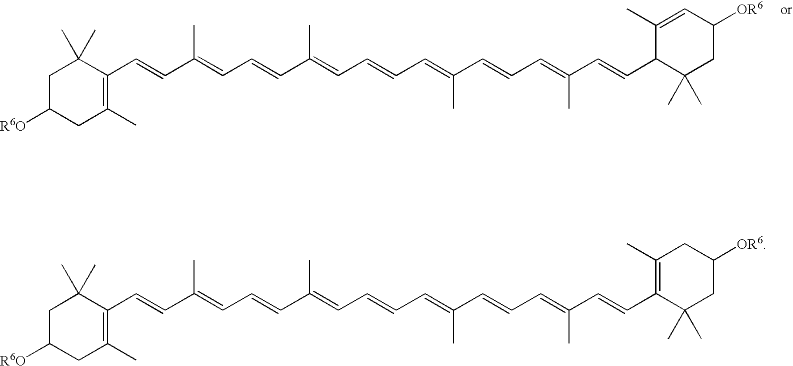
Each —OR⁶ may be independently: 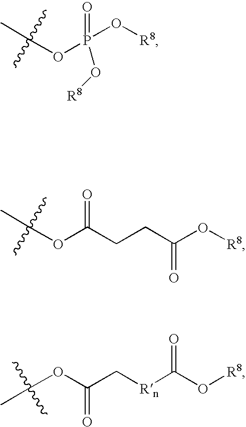
or a co-antioxidant. Each R⁸ may be hydrogen, alkyl, aryl, benzyl or a co-antioxidant. R′ may be CH₂. n may be 1 to 9.

In some embodiments, each —OR⁶ may independently include:

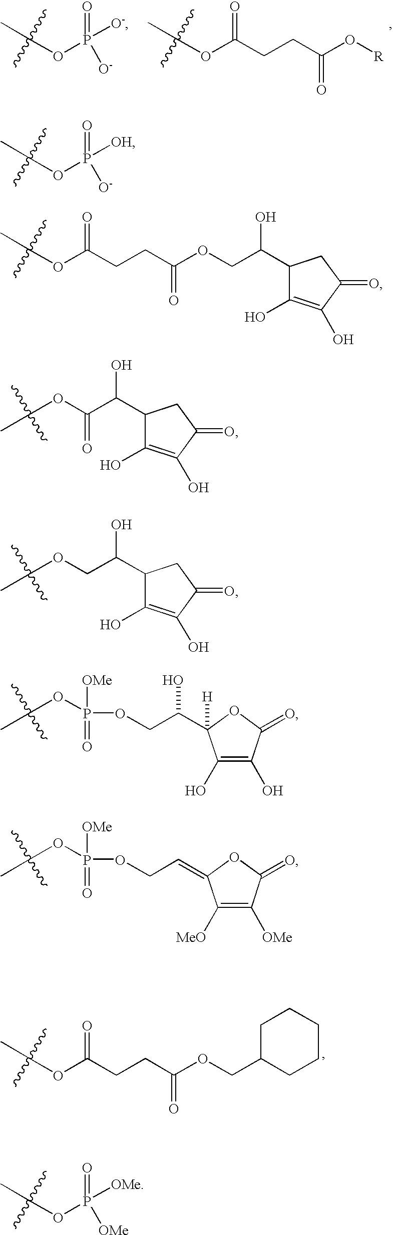

Each R may independently include H, alkyl, aryl, benzyl, Group IA metal, or co-antioxidant.

In some embodiments, each —OR may independently include: 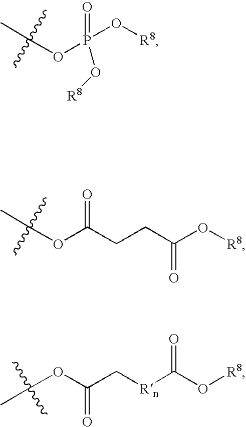
or a co-antioxidant. Each R⁸ may be hydrogen, alkyl, aryl, benzyl, Group IA metal, or a co-antioxidant. R′ may be CH₂. n may be 1 to 9.

In some embodiments, a composition may include one or more carotenoid derivatives or analogs having the structures: 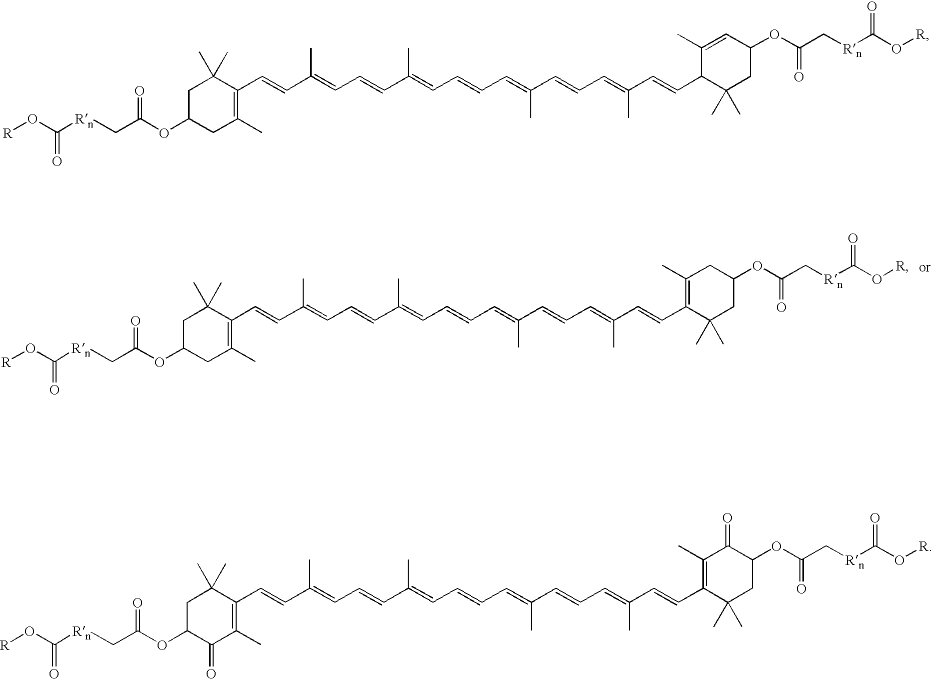
Each R may be independently H, alkyl, aryl, benzyl, Group IA metal, or a co-antioxidant.

In some embodiments, a composition may include one or more carotenoid derivatives or analogs having the structures: 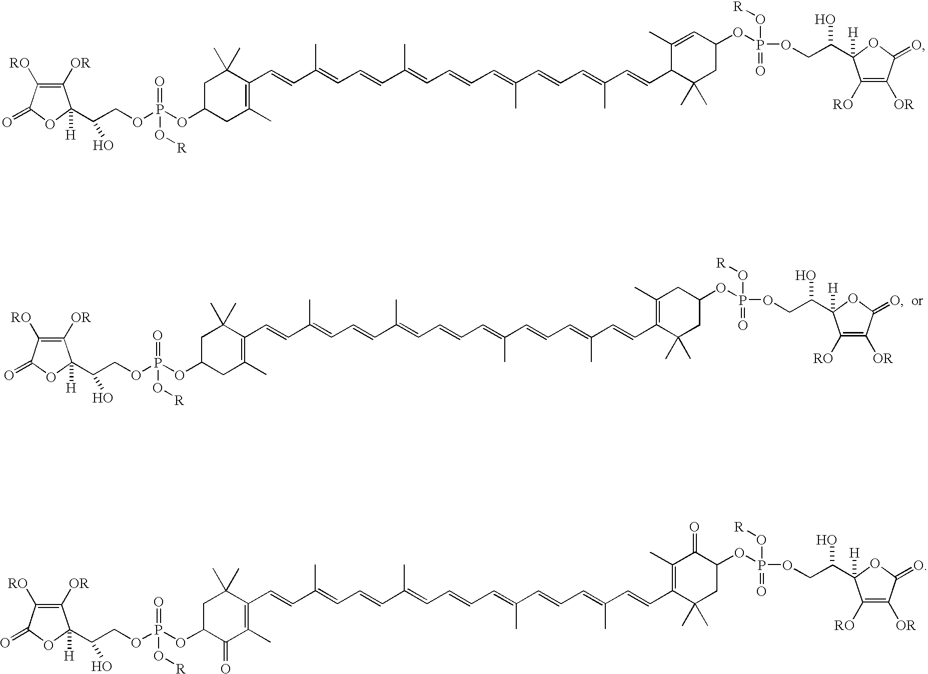
Each R may be independently H, alkyl aryl, benzyl, or a Group IA metal.

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids may include, for example, quercetin, xanthohumol, isoxanthohumol, or genistein.

In some embodiments, a method of treating a visual disability in a subject may include repigmenting a macula of the subject.

In some embodiments, a method of treating a visual disability in a subject may include stabilizing visual acuity of the subject.

In some embodiments, a method of treating a visual disability in a subject may include improving visual acuity of the subject.

In some embodiments, a method of treating a visual disability in a subject may include treating macular degeneration of the subject.

In some embodiments, macular degeneration may include age-related macular degeneration, “wet” macular degeneration, and/or “dry” macular degeneration.

In some embodiments, a carotenoid analog or derivative is an analog or derivative of a naturally occurring carotenoid. In some embodiments, a carotenoid analog or derivative is an analog or derivative of a naturally occurring carotenoid, and wherein the naturally occurring carotenoid is lutein.

In some embodiments, a substituent R⁶ in at least a portion of the carotenoid analogs or derivatives administered to the subject may be cleaved during use. The cleavage product may be biologically active. Cleavage of the carotenoid analog or derivative may be carried out by one or more enzymes.

In some embodiments, a distance between R¹ and R² may be between about 25 Å to about 55 Å. In some embodiments, a distance between R¹ and R² may be between about 40 Å to about 45 Å.

In some embodiments, a composition may include a carotenoid analog or derivative that at least partially dissolves in water.

In some embodiments, a composition may include one or more carotenoid derivatives or analogs which are synthetically derived.

In some embodiments, a composition may be adapted to be administered orally, parenterally, as an aqueous solution, as an aqueous dispersion, intravenously, intravascularly, by intramuscular injection, subcutaneously, and/or transdermally.

In some embodiments, a subject may include a mammal.

In some embodiments, a subject may include a human.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 depicts a graphic representation of several examples of the structures of several xanthophyll carotenoids and synthetic derivatives or analogs that may be used according to some embodiments. (A) astaxanthin; (B) zeaxanthin; (C) lutein; (D) disuccinic acid astaxanthin ester; (E) disodium disuccinic acid astaxanthin ester salt (Cardax™); and (F) divitamin C disuccinate astaxanthin ester; (G) tetrasodium diphosphate astaxanthin ester.

FIG. 2 depicts a time series of the UV/Vis absorption spectra of the disodium disuccinate derivative of natural source lutein in water.

FIG. 3 depicts a UV/Vis absorption spectra of the disodium disuccinate derivative of natural source lutein in water, ethanol, and DMSO.

FIG. 4 depicts a UV/Vis absorption spectra of the disodium disuccinate derivative of natural source lutein in water with increasing concentrations of ethanol.

FIG. 5 depicts a time series of the UV/Vis absorption spectra of the disodium diphosphate derivative of natural source lutein in water.

FIG. 6 depicts a UV/Vis absorption spectra of the disodium diphosphate derivative of natural source lutein in 95% ethanol, 95% DMSO, and water.

FIG. 7 depicts a UV/Vis absorption spectra of the disodium diphosphate derivative of natural source lutein in water with increasing concentrations of ethanol.

FIG. 8 depicts a mean percent inhibition (±SEM) of superoxide anion signal as detected by DEPMPO spin-trap by the disodium disuccinate derivative of natural source lutein (tested in water).

FIG. 9 depicts a mean percent inhibition (±SEM) of superoxide anion signal as detected by DEPMPO spin-trap by the disodium diphosphate derivative of natural source lutein (tested in water).

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Compounds described herein embrace both racemic and optically active compounds. Chemical structures depicted herein that do not designate specific stereochemistry are intended to embrace all possible stereochemistries.

It will be appreciated by those skilled in the art that compounds having one or more chiral center(s) may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound. As used herein, the term “single stereoisomer” refers to a compound having one or more chiral centers that, while it can exist as two or more stereoisomers, are isolated in greater than about 95% excess of one of the possible stereoisomers. As used herein a compound that has one or more chiral centers is considered to be “optically active” when isolated or used as a single stereoisomer.

Definitions

Unless defined otherwise, all technical and scientific terms used herein have the same meaning as commonly understood by one of ordinary skill in the art.

The term “acyl,” as used herein, generally refers to a carbonyl substituent, —C(O)R, where R is alkyl or substituted alkyl, aryl, or substituted aryl, which may be called an alkanoyl substituent when R is alkyl.

The terms “administration,” “administering,” or the like, as used herein, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered by parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

The terms “alkenyl” and “olefin,” as used herein, generally refer to any structure or moiety having the unsaturation C═C. As used herein, the term “alkynyl” generally refers to any structure or moiety having the unsaturation C—C.

The term “alkoxy,” as used herein, generally refers to an —OR group, where R is an alkyl, substituted lower alkyl, aryl, substituted aryl. Alkoxy groups include, for example, methoxy, ethoxy, phenoxy, substituted phenoxy, benzyloxy, phenethyloxy, t-butoxy, and others.

The term “alkyl,” as used herein, generally refers to a chemical substituent containing the monovalent group CₙH₂ₙ, where n is an integer greater than zero. Alkyl includes a branched or unbranched monovalent hydrocarbon radical. An “n-mC” alkyl or “(nC-mC)alkyl” refers to all alkyl groups containing from n to m carbon atoms. For example, a 1-4C alkyl refers to a methyl, ethyl, propyl, or butyl group. All possible isomers of an indicated alkyl are also included. Thus, propyl includes isopropyl, butyl includes n-butyl, isobutyl and t-butyl, and so on. The term alkyl includes substituted alkyls. For example, alkyl includes, but is not limited to: methyl, ethyl, propyl, isopropyl, butyl, iso-butyl, sec-butyl, pentyl, 3-pentyl, hexyl, heptyl, octyl, nonyl, decyl, undecyl, dodecyl, tridecyl, tetradecyl or pentadecyl; “alkenyl” includes but is not limited to vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl; 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, 1-tridecenyl, 2-tridecenyl, 3-tridecenyl, 4-tridecenyl, 5-tridecenyl, 6-tridecenyl, 7-tridecenyl, 8-tridecenyl, 9-tridecenyl, 10-tridecenyl, 11-tridecenyl, 12-tridecenyl, 1-tetradecenyl, 2-tetradecenyl, 3-tetradecenyl, 4-tetradecenyl, 5-tetradecenyl, 6-tetradecenyl, 7-tetradecenyl, 8-tetradecenyl, 9-tetradecenyl, 10-tetradecenyl, 11-tetradecenyl, 12-tetradecenyl, 13-tetradeceny, 1-pentadecenyl, 2-pentadecenyl, 3-pentadecenyl, 4-pentadecenyl, 5-pentadecenyl, 6-pentadecenyl, 7-pentadecenyl, 8-pentadecenyl, 9-pentadecenyl, 10-pentadecenyl, 11-pentadecenyl, 12-pentadecenyl, 13-pentadecenyl, 14-pentadecenyl; “alkoxy” includes but is not limited to methoxy, ethoxy, propoxy, isopropoxy, butoxy, iso-butoxy, sec-butoxy, pentoxy, 3-pentoxy, hexoxy, heptyloxy, octyloxy, nonyloxy, decyloxy, undecyloxy, dodecyloxy, tridecyloxy, tetradecyloxy, or pentadecyloxy.

The term “amino,” as used herein, generally refers to a group —NRR′, where R and R′ may independently be hydrogen, lower alkyl, substituted lower alkyl, aryl, substituted aryl or acyl.

The terms “amphiphile” or “amphiphilic,” as used herein, refer to a molecule or species, which exhibits both hydrophilic and lipophilic character. In general, an amphiphile contains a lipophilic moiety and a hydrophilic moiety. The terms “lipophilic” and “hydrophobic” are interchangeable as used herein. An amphiphile may form a Langmuir film. An amphiphile may be surface-active in solution. A bolaamphiphile is a special case in which the hydrophobic spacer is substituted on each end with a hydrophilic moiety.

Non-limiting examples of hydrophobic groups or moieties include lower alkyl groups, alkyl groups having 7, 8, 9, 10, 11, 12, or more carbon atoms, including alkyl groups with 14-30, or 30 or more carbon atoms, substituted alkyl groups, alkenyl groups, alkynyl groups, aryl groups, substituted aryl groups, saturated or unsaturated cyclic hydrocarbons, heteroaryl, heteroarylalkyl, heterocyclic, and corresponding substituted groups. A hydrophobic group may contain some hydrophilic groups or substituents insofar as the hydrophobic character of the group is not outweighed. In further variations, a hydrophobic group may include substituted silicon atoms, and may include fluorine atoms. The hydrophobic moieties may be linear, branched, or cyclic.

Non-limiting examples of hydrophilic groups or moieties include hydroxyl, methoxy, phenyl, carboxylic acids and salts thereof, methyl, ethyl, and vinyl esters of carboxylic acids, amides, amino, cyano, isocyano, nitrile, ammonium salts, sulfonium salts, phosphonium salts, mono- and di-alkyl substituted amino groups, polypropyleneglycols, polyethylene glycols, epoxy groups, acrylates, sulfonamides, nitro, —OP(O)(OCH₂CH₂N⁺RRR)O⁻, guanidinium, aminate, acrylamide, pyridinium, piperidine, and combinations thereof, wherein each R is independently selected from H or alkyl. Further examples include polymethylene chains substituted with alcohol, carboxylate, acrylate, or methacrylate. Hydrophilic moieties may also include alkyl chains having internal amino or substituted amino groups, for example, internal —NH—, —NC(O)R—, or —NC(O)CH═CH₂-groups, wherein R is H or alkyl. Hydrophilic moieties may also include polycaprolactones, polycaprolactone diols, poly(acetic acid)s, poly(vinyl acetates)s, poly(2-vinyl pyridine)s, cellulose esters, cellulose hydroxylethers, poly(L-lysine hydrobromide)s, poly(itaconic acid)s, poly(maleic acid)s, poly(styrenesulfonic acid)s, poly(aniline)s, or poly(vinyl phosphonic acid)s. A hydrophilic group may contain some hydrophobic groups or substituents insofar as the hydrophilic character of the group is not outweighed.

The term “analog,” as used herein, generally refers to a compound that resembles another in structure but is not necessarily an isomer.

The term “antioxidant,” as used herein, generally refers to any of various substances (e.g., beta-carotene, vitamin C, vitamin E, flavonoids, polyphenolics, and alpha-tocopherol) that inhibit oxidation or reactions promoted by oxygen and peroxides and that include many held to protect the living body from the deleterious effects of free radicals.

The term “aryl,” as used herein, generally refers to a chemical substituent containing an aromatic group. An aromatic group may be a single aromatic ring or multiple aromatic rings that are fused together, coupled covalently, or coupled to a common group such as a methylene, ethylene, or carbonyl, and includes polynuclear ring structures. An aromatic ring or rings may include, but is not limited to, substituted or unsubstituted phenyl, naphthyl, biphenyl, diphenylmethyl, and benzophenone groups. The term “aryl” includes substituted aryls.

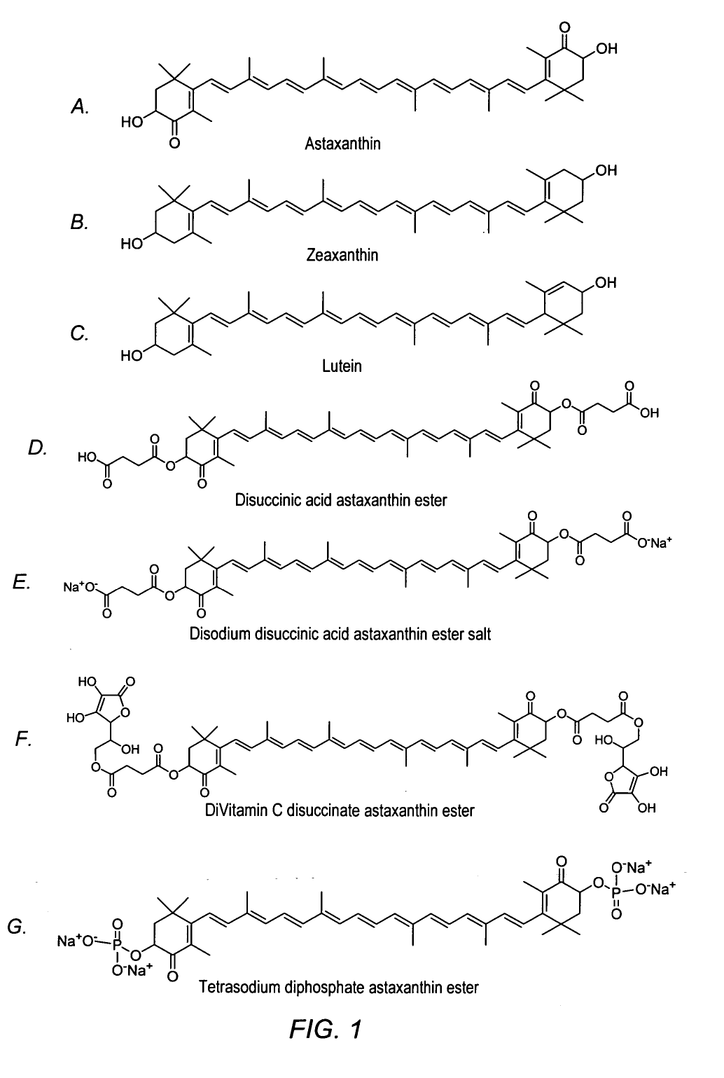

The terms such as “carotenoid analog” and “carotenoid derivative,” as used herein, generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically in FIG. 1, D-G. “Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

The term “co-antioxidant,” as used herein, generally refers to an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative potential of one or more anti-oxidants acting synergistically may be greater than the sum of the oxidative potential of each component anti-oxidant).

The terms “coupling” and “coupled,” as used herein, with respect to molecular moieties or species, atoms, synthons, cyclic compounds, and nanoparticles refers to their attachment or association with other molecular moieties or species, atoms, synthons, cyclic compounds, and nanoparticles. The attachment or association may be specific or non-specific, reversible or non-reversible, the result of chemical reaction, or complexation or charge transfer. The bonds formed by a coupling reaction are often covalent bonds, or polar-covalent bonds, or mixed ionic-covalent bonds, and may sometimes be Coulombic forces, ionic or electrostatic forces or interactions.

The term “cycloalkyl,” as used herein, includes, but is not limited to, cyclopropyl, cyclobutyl, cyclopentyl, cyclohexyl, cycloheptyl, or cyclooctyl.

The term “derivative,” as used herein, generally refers to a chemical substance derived from another substance either directly or by modification or partial substitution.

The term “functionalized,” as used herein, generally refers to the presence of a reactive chemical moiety or functionality. A functional group may include, but is not limited to, chemical groups, biochemical groups, organic groups, inorganic groups, organometallic groups, aryl groups, heteroaryl groups, cyclic hydrocarbon groups, amino (—NH₂), hydroxyl (—OH), cyano (—C≡N), nitro (NO₂), carboxyl (—COOH), formyl (—CHO), keto (—CH₂C(O)CH₂—), ether (—CH₂—O—CH₂—), thioether (—CH₂—S—CH₂—), alkenyl (—C═C—), alkynyl, (—C≡C—), epoxy (e.g., 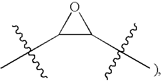
metalloids (functionality containing Si and/or B) and halo (F, Cl, Br, and I) groups. In some embodiments, the functional group is an organic group.

The term “heteroaryl,” as used herein, generally refers to a completely unsaturated heterocycle.

The term “heterocycle,” as used herein, generally refers to a closed-ring structure, in which one or more of the atoms in the ring is an element other than carbon. Heterocycle may include aromatic compounds or non-aromatic compounds. Heterocycles may include rings such as thiophene, pyridine, isoxazole, phthalimide, pyrazole, indole, furan, or benzo-fused analogs of these rings. Examples of heterocycles include tetrahydrofuran, morpholine, piperidine, pyrrolidine, and others. In some embodiments, “heterocycle” is intended to mean a stable 5- to 7-membered monocyclic or bicyclic or 7- to 10-membered bicyclic heterocyclic ring which is either saturated or unsaturated, and which consists of carbon atoms and from 1 to 4 heteroatoms (e.g., N, O, and S) and wherein the nitrogen and sulfur heteroatoms may optionally be oxidized, and the nitrogen may optionally be quaternized, and including any bicyclic group in which any of the above-defined heterocyclic rings is fused to a benzene ring. In some embodiments, heterocycles may include cyclic rings including boron atoms. The heterocyclic ring may be attached to its pendant group at any heteroatom or carbon atom that results in a stable structure. The heterocyclic rings described herein may be substituted on carbon or on a nitrogen atom if the resulting compound is stable. Examples of such heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzofuranyl, benzothiophenyl, carbazole, chromanyl, chromenyl, cinnolinyl, decahydroquinolinyl, furanyl, furazanyl, imidazolidinyl, imidazolinyl, imidazolyl, indolinyl, indolizinyl, indolyl, isobenzofuranyl, isochromanyl, isoindolinyl, isoindolyl, isoquinolinyl (benzimidazolyl), isothiazolyl, isoxazolyl, morpholinyl, naphthyridinyl, octahydroisoquinolinyl, oxazolidinyl, oxazolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, pyrrolyl, quinazolinyl, quinolinyl, quinoxalinyl, quinuclidinyl, carbolinyl, tetrahydrofuranyl, tetrahydroisoquinolinyl, tetrahydroquinolinyl, tetrazolyl, thianthrenyl, thiazolyl, thienyl, thiophenyl, triazinyl, xanthenyl. Also included are fused ring and spiro compounds containing, for example, the above heterocycles.

The terms “in need of treatment” or “in need thereof,” as used herein, when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

The term “ion,” as used herein, generally refers to an atom(s), radical, or molecule(s) that has lost or gained one or more electrons and has thus acquired an electric charge.

The term “microbe,” as used herein, generally refers to a minute life form; a microorganism. In some embodiments, a microbe may include a bacterium that causes disease.

The term “nutraceuticals,” as used herein, generally refers to dietary supplements, foods, or medical foods that: 1. possess health benefits generally defined as reducing the risk of a disease or health condition, including the management of a disease or health condition or the improvement of health; and 2. are safe for human consumption in such quantity, and with such frequency, as required to realize such properties.

The terms “oligomeric” and “polymeric,” as used herein, are used interchangeably herein to generally refer to multimeric structures having more than one component monomer or subunit.

The term “organ,” as used herein, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

Terms such as “pharmaceutical composition,” “pharmaceutical formulation,” “pharmaceutical preparation,” or the like, as used herein, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof: Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

The term “pharmaceutically acceptable salts,” as used herein, includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The terms “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

The term “pharmacologically inert,” as used herein, generally refers to a compound, additive, binder, vehicle, and the like, that is substantially free of any pharmacologic or “drug-like” activity.

The term “polymerizable element,” as used herein, generally refers to a chemical substituent or moiety capable of undergoing a self-polymerization and/or co-polymerization reaction (e.g., vinyl derivatives, butadienes, trienes, tetraenes, diolefins, acetylenes, diacetylenes, styrene derivatives).

The phrase “prophylactically effective amount,” as used herein, generally refers to an amount of a pharmaceutical composition that will substantially prevent, delay or reduce the risk of occurrence of the biological or physiological event in a cell, a tissue, a system, animal or human that is being sought by a researcher, veterinarian, physician or other caregiver.

The terms “Rⁿ,” as used herein, in a chemical formula refer to hydrogen or a functional group, each independently selected, unless stated otherwise. In some embodiments the functional group may be an organic group. In some embodiments the functional group may be an alkyl group. In some embodiment, the functional group may be a hydrophobic or hydrophilic group.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “subject,” as used herein, may be generally defined as all mammals, in particular humans.

The term “substituted alkyl,” as used herein, generally refers to an alkyl group with an additional group or groups attached to any carbon of the alkyl group. Substituent groups may include one or more functional groups such as alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbons, heterocycles, and other organic groups.

The term “substituted aryl,” as used herein, generally refers to an aryl group with an additional group or groups attached to any carbon of the aryl group. Additional groups may include one or more functional groups such as lower alkyl, aryl, acyl, halogen, alkylhalo, hydroxy, amino, alkoxy, alkylamino, acylamino, acyloxy, aryloxy, aryloxyalkyl, thioether, heterocycles, both saturated and unsaturated cyclic hydrocarbons which are fused to the aromatic ring(s), coupled covalently or coupled to a common group such as a methylene or ethylene group, or a carbonyl coupling group such as in cyclohexyl phenyl ketone, and others.

The term “substituted heterocycle,” as used herein, generally refers to a heterocyclic group with an additional group or groups attached to any element of the heterocyclic group. Additional groups may include one or more functional groups such as lower alkyl, aryl, acyl, halogen, alkylhalos, hydroxy, amino, alkoxy, alkylamino, acylamino, acyloxy, aryloxy, aryloxyalkyl, thioether, heterocycles, both saturated and unsaturated cyclic hydrocarbons which are fused to the heterocyclic ring(s), coupled covalently or coupled to a common group such as a methylene or ethylene group, or a carbonyl coupling group such as in cyclohexyl phenyl ketone, and others.

The term “substrate,” as used herein, generally refers to a body or base layer or material (e.g., onto which other layers are deposited).

The phrase “the synergistic combination of more than one structural analog or derivative or synthetic intermediate of carotenoids,” as used herein, may be generally defined as any composition including one structural carotenoid analog or derivative or synthetic intermediate combined with one or more other structural carotenoid analogs or derivatives or synthetic intermediate or co-antioxidants, either as derivatives or in solutions and/or formulations.

The phrase “therapeutically effective amount,” as used herein, generally refers to an amount of a drug or pharmaceutical composition that will elicit at least one desired biological or physiological response of a cell, a tissue, a system, animal or human that is being sought by a researcher, veterinarian, physician or other caregiver.

The term “thioether,” as used herein, generally refers to the general structure R—S—R′ in which R and R′ are the same or different and may be alkyl, aryl or heterocyclic groups. The group —SH may also be referred to as “sulfhydryl” or “thiol” or “mercapto.”

The term “tissue,” as used herein, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

The term “visual acuity,” as used herein, generally refers to the relative ability of the visual organ to resolve detail that is usually expressed as the reciprocal of the minimum angular separation in minutes of two lines just resolvable as separate and that forms in the average human eye an angle of one minute.

The term “visual disability,” as used in, generally refers to any condition which detrimentally effects vision or eyesight. Conditions may be due to disease and/or to natural deterioration of vision associated with age.

The term “xanthophyll carotenoid,” as used herein, generally refers to a naturally occurring or synthetic 40-carbon polyene chain with a carotenoid structure that contains at least one oxygen-containing functional group. The chain may include terminal cyclic end groups. Exemplary, though non-limiting, xanthophyll carotenoids include astaxanthin, zeaxanthin, lutein, echinenone, canthaxanthin, and the like. Non-limiting examples of carotenoids that are not xanthophyll carotenoids include β-carotene and lycopene.

Treatment of Visual Disabilities

Recent studies have demonstrated the utility of lutein-based supplementation for the clinical improvement of vision, reduction of ultraviolet (UV)-based inflammation, and potentially the inhibition and/or amelioration of age-related macular degeneration (ARMD). The potential utility of lutein- and zeaxanthin-based formulations as well as other carotenoids may be extended for clinical application by providing compounds with sufficient aqueous dispersibility. Aqueous dispersibility may allow for parenteral administration of carotenoid analogs or derivatives. Parenteral administration may allow for better treating the significant human population of carotenoid oral non-responders as well as acute clinical application(s) requiring rapid loading of therapeutic doses. The administration of these compounds may provide a stabilization and/or increase of macular pigment density, with improvement of refractive index and chromatic aberration resulting from said MPOD stabilization and/or increases. This may provide immediate or near-immediate positive effects on visual acuity. This may also provide immediate or near-immediate effects on measures of visual function. The direct superoxide anion scavenging ability of the carotenoid analogs and derivatives described herein may provide further health benefits.

In some embodiments, carotenoid analogs or derivatives may be employed to stabilize and/or improve visual acuity. In some embodiments, carotenoid analogs or derivatives may be employed to inhibit and/or ameliorate visual disabilities. Carotenoid analogs or derivatives may have increased water solubility and/or water dispersibility relative to some or all known naturally occurring carotenoids.

In some embodiments, carotenoid analogs or derivatives may be employed in “self-formulating” aqueous solutions, in which the compounds spontaneously self-assemble into macromolecular complexes. These complexes may provide stable formulations in terms of shelf-life. The same formulations may be parenterally administered, upon which the spontaneous self-assembly is overcome by interactions with serum and/or tissue components in vivo.

Some specific embodiments may include phosphate, succinate, co-antioxidant (e.g., Vitamin C, Vitamin C analogs, Vitamin C derivatives, Vitamin E, Vitamin E analogs, Vitamin E derivatives, or flavonoids), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, one or more co-antioxidants may be coupled to a carotenoid or carotenoid derivative or analog.

In some embodiments, visual disabilities may include diagnosis of atrophic ARMD (ICD9 362.51) by stereo bio-ophthalmoscopy and/or at least one vision-degrading visual-psychophysical abnormality associated with ARMD in one or both eyes. The latter may include depressed contrast sensitivity (CSF) at one or more of four spatial frequencies (3, 6, 12, or 18 cc/degree) not attributable to cataract or ocular disease; an abnormal photo-stress glare recovery (GR) deficit or deficits of the Amsler grid (i.e., intermittent/permanent visual spots [scotomas]); and/or distortions of lines (metamorphopsia). Visual disabilities may include clear non-lenticular ocular media (cornea, aqueous, and vitreous), free of advanced glaucoma and diabetes or any other ocular or systemic disease that could affect central or parafoveal macular visual function.

Determination of visual acuity and/or the extent of visual disabilities may be measured by a variety of known methods. Methods may be used to determine baseline for a subject or group of subjects as well as rate and extent of progress.

In some embodiments, baseline and final ocular lens opacification (cataract) may be evaluated subjectively by a single observer, using a Haag-Streit slit-lamp biomicroscope. A 7-increment validated Lens Opacity Cataract Scale (LOCSIII) photographic transparency may be placed on a 5000 K color temperature, 8-watt fluorescent photographic light box, according to standard procedures. Retinal images may be taken with a Canon model CF-600 Vi Fundus camera at baseline and study completion, and may be rated by one or more retinologists. MPOD may be measured by heterochromatic flicker photometry using a MacularMetrics® instrument (Rehoboth, Mass.), according to the manufacturers' protocol. It may be determined by subjects matching a one-degree 460/540 nanometer flickering stimulus for perceived brightness foveally and at a retinal location seven degrees extra-foveally, where the MPOD is virtually zero.

Refractive error may be neutralized with lenses prior to acuity and glare testing at each visit, and resultant trial lenses may be used for all vision tests. Glare recovery (GR) is a measure of both macular function and retinal health. Subjects may monocularly view a 6500 lux, 5000 K color temperature light box held at 40 cm from their eye for one minute pre-adaptation. Following this visual stress, the subject may be asked to attempt to read a superthreshold low-contrast line of print. Examiners may monitor accuracy and time required for macular function to return to normal following the photo-stressed condition. One minute or less to recovery is normal.

Monocular visual acuity at distance may be measured via random presentation of distance Snellen letters on a Mentor Opththalmics BVAT-II® computer screen under mesopic lighting conditions. Low-vision patients with Snellen acuity less than 20/200 may be tested with a Designs for Vision® Feinbloom distance acuity test for the partially sighted. In both cases, fractional Snellen acuity may be converted to Log minimal angle of resolution (LogMAR).

Visual acuity at near may be measured for each eye using low- and high-contrast SKILL test targets. Contrast sensitivity function (CSF) measures the least amount of contrast needed to detect visual stimuli at different spatial frequencies. It identifies selective retinal deficits in visual processing at an earlier stage than is possible with conventional visual acuity testing. The Vector Vision CSV 1000® contrast sensitivity test system may be used.

For low-vision patients, the CSV 1000L-V Pelli-Robson type single large letter chart of varying contrasts may be used. Activities of daily living, night driving, and glare recovery symptoms may be evaluated on a 4- to 20-point VFQ-14 rating scale used by the National Eye Institute.

Subjects may be provided an integrated instruction sheet/questionnaire/Amsler grid to monitor changes in vision over time. Subjects at each visit may be asked if they felt their overall vision had worsened, remained the same, or improved, and the number of boxes missing (scotomas) or distorted (metamorphopsia) may be counted. Reports of vision change may be digitally videotaped.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their biovailability and hence proper evaluation of efficacy. Esterification or etherification may be useful to increase oral bioavailabilty, a fortuitous side effect of the esterification process which can increase solubility in gastric mixed micelles. The net overall effect is an improvement in potential clinical utility for the lipophilic carotenoid compounds as therapeutic agents.

In some embodiments, the principles of retrometabolic drug design may be utilized to produce novel soft drugs from the asymmetric parent carotenoid scaffold (e.g., RRR-lutein (β,ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids. Previous research has demonstrated lutein alone administered to subjects increased mean eye macular pigment optical density as well as Snellen equivalent visual acuity. Lutein administered in combination with a broad range of minerals and antioxidants to subjects increased mean eye macular pigment optical density as well as Snellen equivalent visual acuity. However, the previous research demonstrated that lutein alone showed greater improvements than lutein in combination with a broad range of minerals and antioxidants as demonstrated in Richer et al “Double-masked, placebo-controlled, randomized trial of lutein and antioxidant supplementation in the intervention of atrophic age-related macular degeneration: the Veterans LAST study (Lutein Antioxidant Supplementation Trial)” OPTOMETRY Vol. 75, No. 4 Apr. 2004 which is incorporated by reference as if fully set forth herein. Contradictory to previous research is improved results accomplished with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

Carotenoids and the Preparation and use Thereof

In some embodiments, a composition may include one or more carotenoids. Carotenoids may include carotenes and xanthophyll carotenoids. In some embodiments, carotenoids may have the general structure: 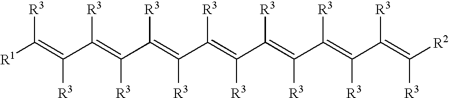
where each R³ is independently hydrogen or methyl, and where each R¹ and R² are independently: 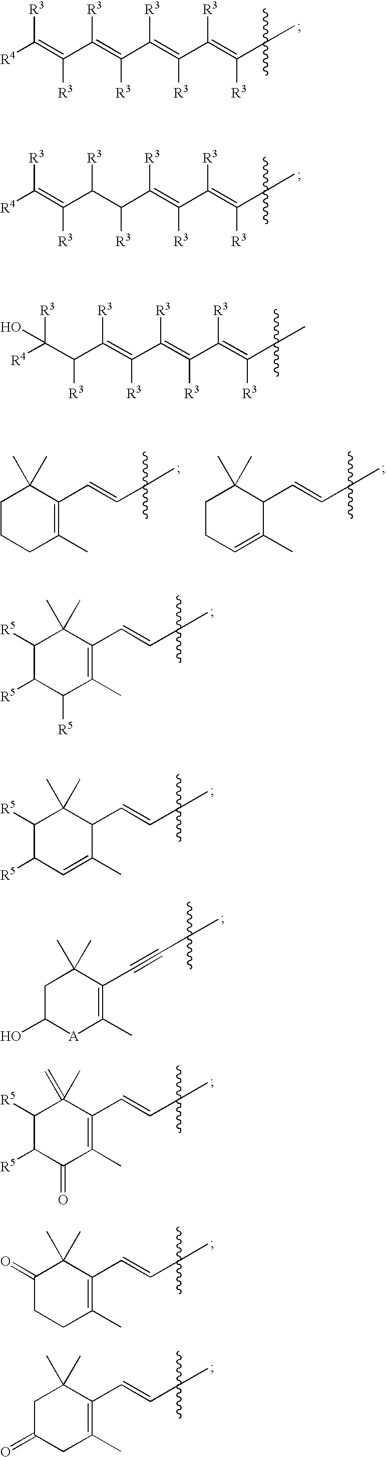 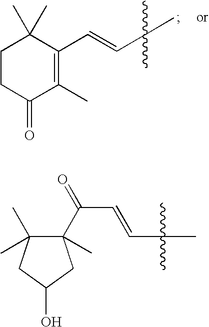
where R⁴ is hydrogen, methyl, or —CH₂OH; and where each R⁵ is independently hydrogen or —OH. Sources of some of these carotenoids can be found, for example, in the reference “Key to Carotenoids”, Otto Straub, 2^{nd} Ed., Birkhauser Verlag, Boston, 1987, which is incorporated herein by reference.

In some embodiments, carotenoids may have the general structure: 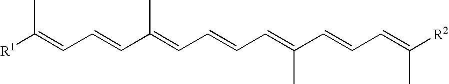
where each R¹ and R² are independently: 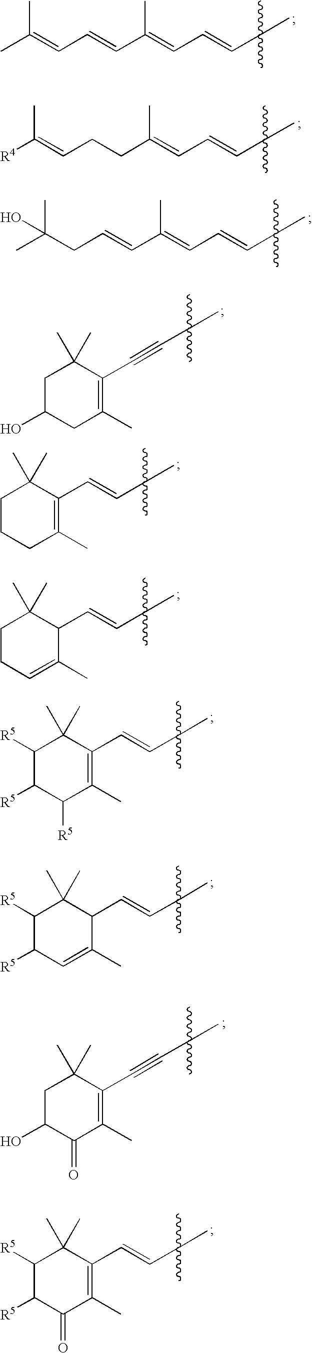 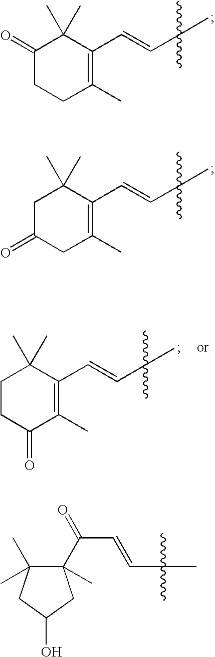
where R⁴ is hydrogen, methyl, or —CH₂OH; and where each R⁵ is independently hydrogen or —OH.

In some embodiments, carotenoids may include xanthophyll carotenoids having the general structure: 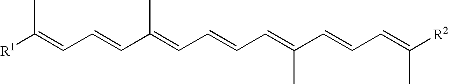
where each R¹ and R² are independently: 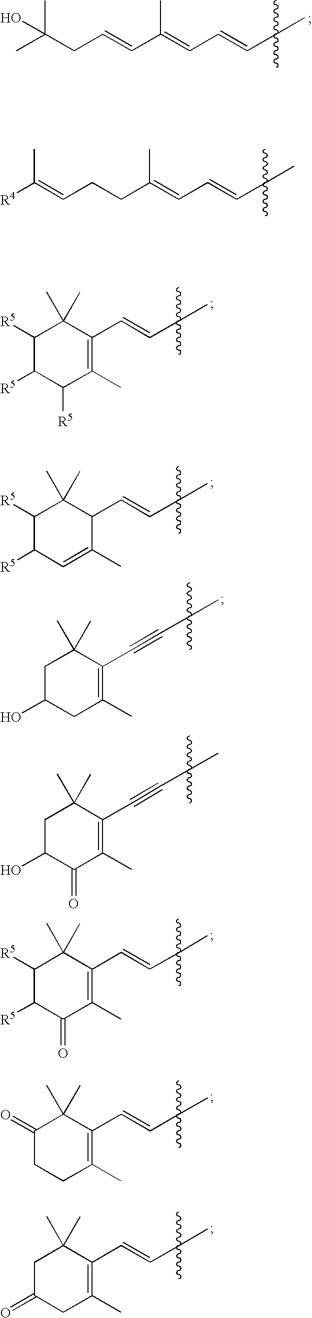 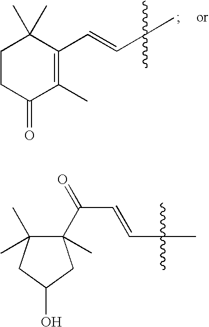
where R⁴ is —CH₂—OH; and where each R⁵ is independently hydrogen or —OH.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, flavonoids, flavonoid analogs, or flavonoid derivatives), or combinations thereof derivatives or analogs of carotenoids. Flavonoids may include, for example, quercetin, xanthohumol, isoxanthohumol, or genistein. Vitamin E may generally be divided into two categories including tocopherols having a general structure 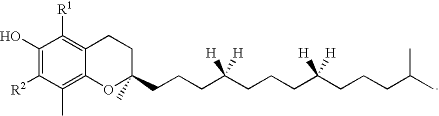
Alpha-tocopherol is used to designate when R¹═R²═CH₃. Beta-tocopherol is used to designate when R¹═CH₃ and R²═H. Gamma-tocopherol is used to designate when R¹═H and R²═CH₃. Delta-tocopherol is used to designate when R¹═R²═H.

The second category of Vitamin E may include tocotrienols having a general structure 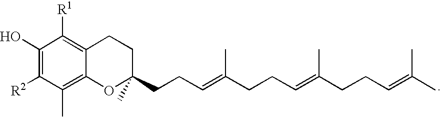
Alpha-tocotrienol is used to designate when R¹═R²═CH₃. Beta-tocotrienol is used to designate when R¹═CH₃ and R²═H. Gamma-tocotrienol is used to designate when R¹═H and R²═CH₃. Delta-tocotrienol is used to designate when R¹═R²═H.

Quercetin, a flavonoid, has the structure 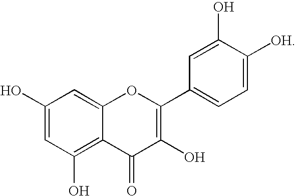
In some embodiments, one or more co-antioxidants may be coupled to a carotenoid or carotenoid derivative or analog. Derivatives of one or more carotenoid analogs may be formed by coupling one or more free hydroxy groups of the co-antioxidant to a portion of the carotenoid.

Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

In some embodiments, a chemical compound including a carotenoid derivative or analog may have the general structure: 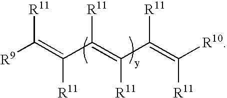
Each R¹¹ may be independently hydrogen or methyl. R⁹ and R¹⁰ may be independently H, an acyclic alkene with one or more substituents, or a cyclic ring including one or more substituents. y may be 5 to 12. In some embodiments, y may be 3 to 15. In certain embodiments, the maximum value of y may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be included in a pharmaceutical composition.

In some embodiments, a method of treating a visual disability may include administering to the subject an effective amount of a pharmaceutically acceptable composition including one or more synthetic analogs or derivatives of a carotenoid. The synthetic analog or derivative of the carotenoid may have the structure 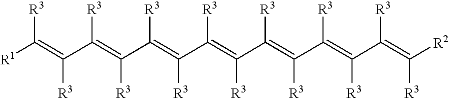
where each R³ is independently hydrogen or methyl, and where each R¹ and R² are independently: 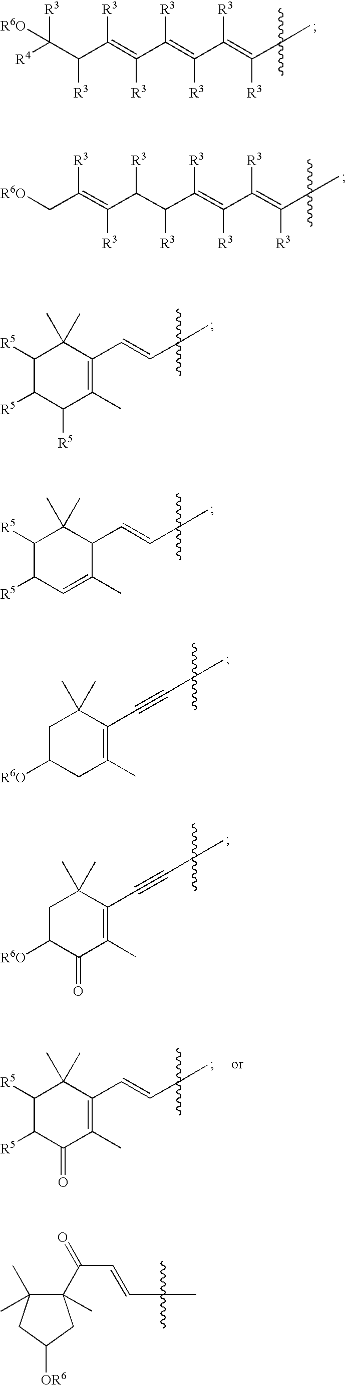
where R⁴ is hydrogen or methyl; where each R⁵ is independently hydrogen, —OH, or —OR⁶ wherein at least one R⁵ group is —OR⁶; wherein each R⁶ may be independently: alkyl; aryl; -alkyl-N(R⁷)₂; -aryl-N(R⁷)₂; -alkyl-N(R⁷)₃; -aryl-N⁺(R⁷)₃; -alkyl-CO₂R⁷; -aryl-CO₂R⁷; -alkyl-CO₂—; -aryl-CO₂—; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with a visual disability.

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

In some embodiments, a method of inhibiting or reducing at least some of the side effects associated with a selective visual disability may include administering to the subject an effective amount of a pharmaceutically acceptable formulation including one or more synthetic analogs or derivatives of a carotenoid. The synthetic analog or derivative of the carotenoid may have the structure 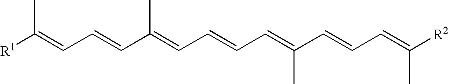
where each R¹ and R² are independently: 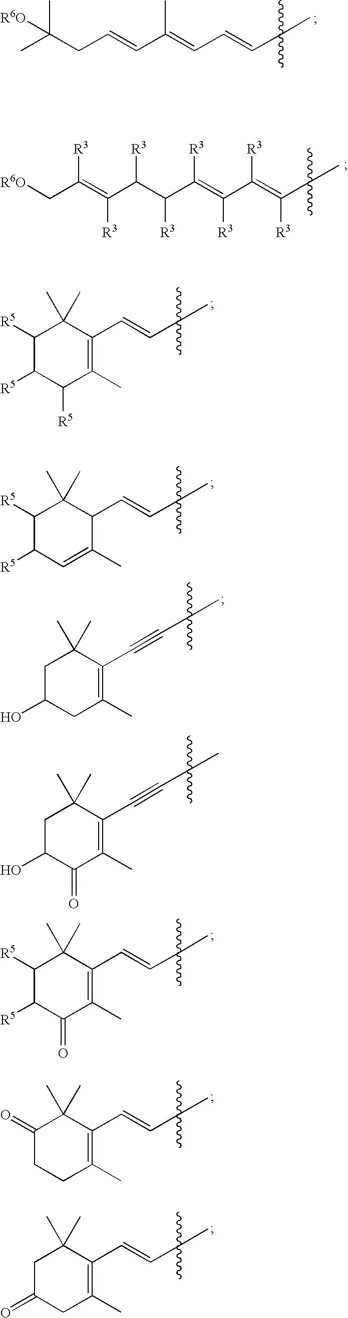 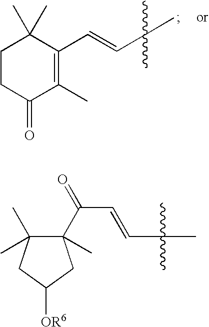
where each R⁵ is independently hydrogen, —OH, or —OR⁶ wherein at least one R⁵ group is —OR⁶; wherein each R⁶ may be independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂—; -aryl-CO₂—; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with a visual disability.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 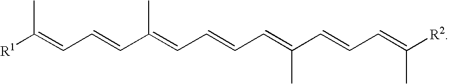
Each R¹ and R² may be independently: 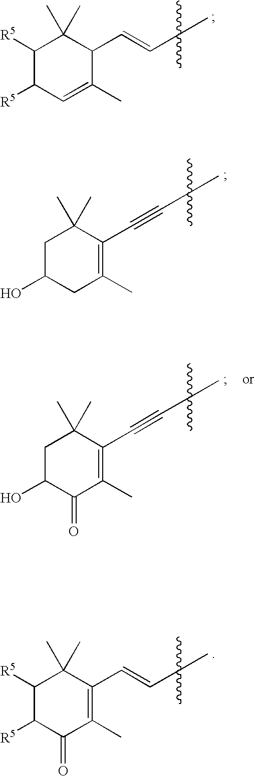
Each R⁵ may be independently hydrogen, —OH, or —OR⁶. At least one R⁵ group may be —OR⁶. Each R⁶ may be independently: 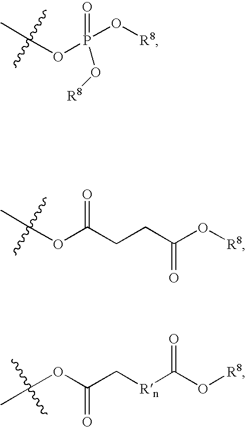
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 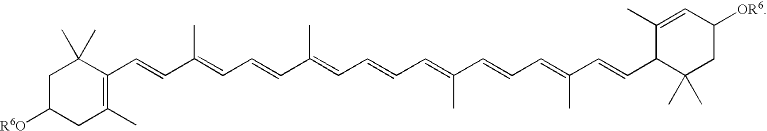
Each —OR⁶ may be independently: 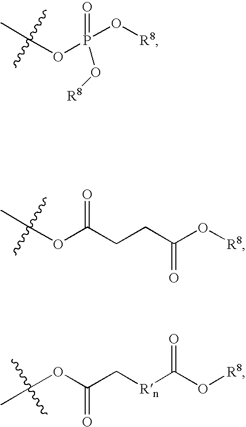
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 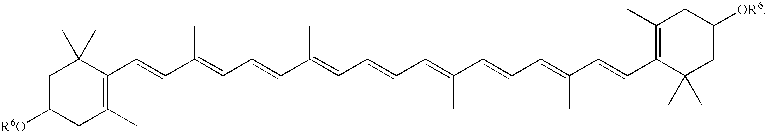
Each —OR⁶ may be independently: 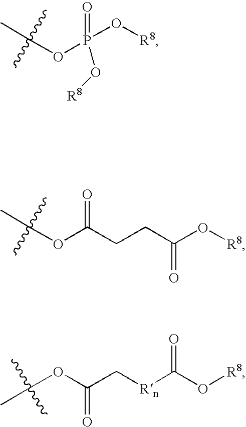
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including two or more carotenoid derivatives or analogs. The analog or derivative of the carotenoid may have the structure 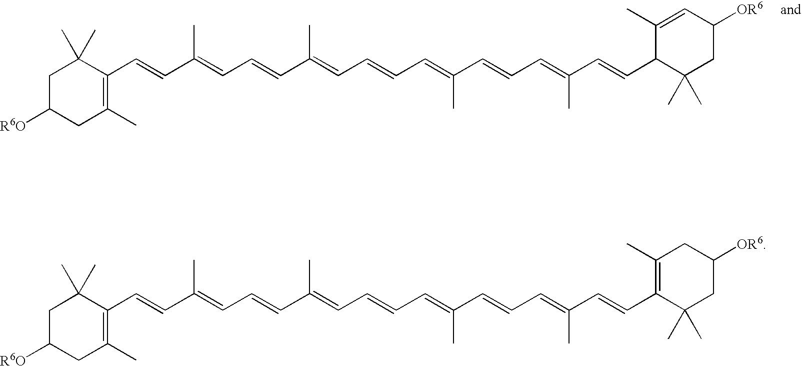
Each —OR⁶ may be independently: 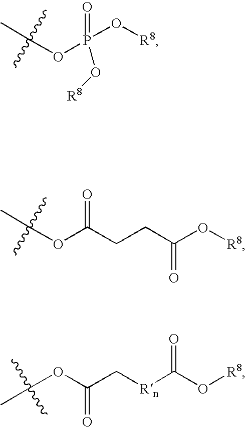
or a co-antioxidant. Each R⁸ may be hydrogen, alkyl, aryl, benzyl or a co-antioxidant. R′ may be CH₂. n may be 1 to 9.

In some embodiments, a method of treating a visual disability in a subject may include administering to the subject an effective amount of a pharmaceutically acceptable composition or formulation including one or more carotenoid derivatives or analogs. The analog or derivative of the carotenoid may have the structure 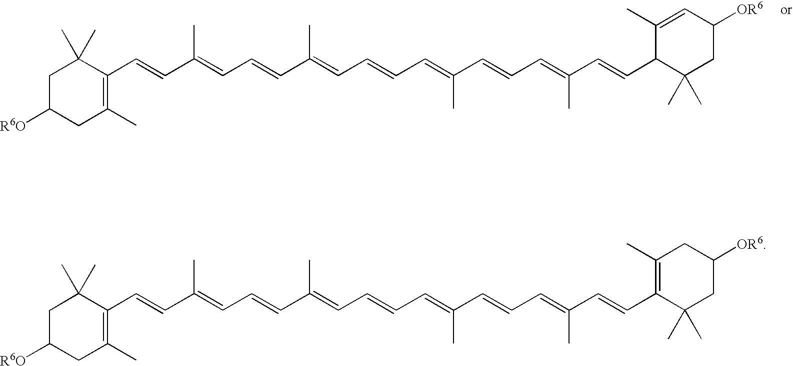
Each —OR⁶ may be independently: 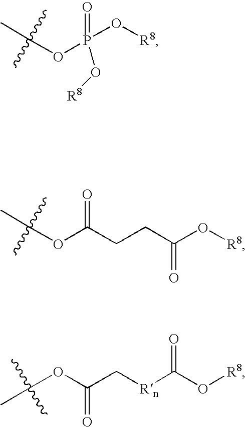
or a co-antioxidant. Each R⁸ may be hydrogen, alkyl, aryl, benzyl or a co-antioxidant. R′ may be CH₂. n may be 1 to 9.

In some embodiments, each —OR⁶ may independently include: 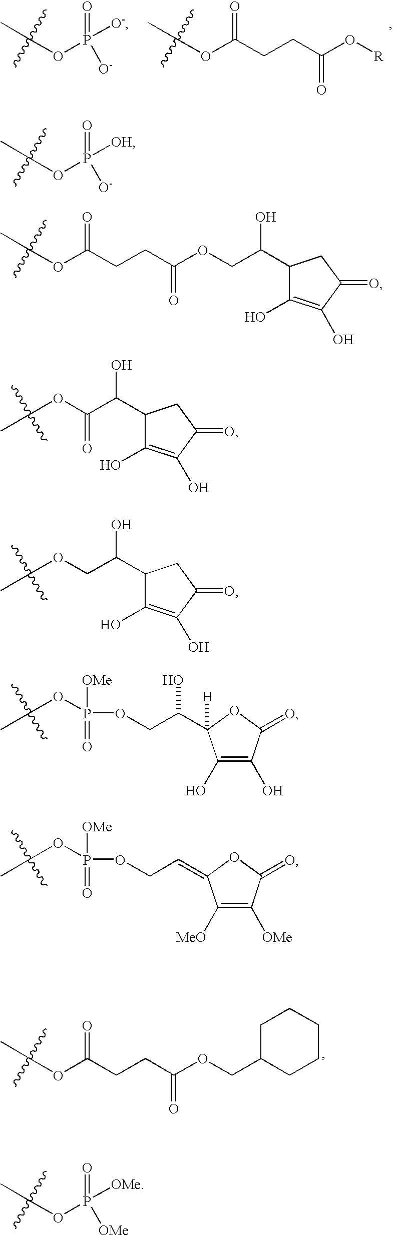
Each R may independently include H, alkyl, aryl, benzyl, Group IA metal, or co-antioxidant.

In some embodiments, each —OR⁶ may independently include: 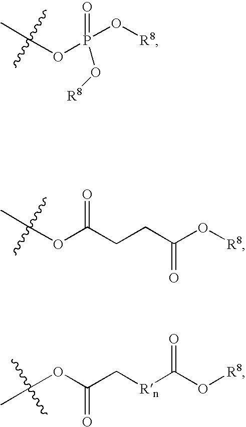
or a co-antioxidant. Each R⁸ may be hydrogen, alkyl, aryl, benzyl, Group IA metal, or a co-antioxidant. R′ may be CH₂. n may be 1 to 9.

When R⁶ is an amino acid derivative or a peptide, coupling of the amino acid or the peptide is accomplished through an ester linkage. The ester linkage may be formed between a free hydroxyl of the xanthophyll carotene and the carboxylic acid of the amino acid or peptide. When R⁹ is an amino acid derivative or a peptide, coupling of the amino acid or the peptide is accomplished through an amide linkage. The amide linkage may be formed between the terminal carboxylic acid group of the linker attached to the xanthophyll carotene and the amine of the amino acid or peptide.

When R⁶ is a sugar, R⁶ includes, but is not limited to the following side chains:

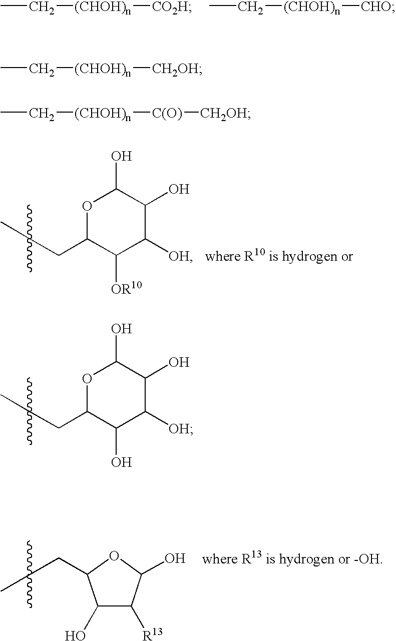

When R⁶ is a nucleoside, R⁶ may have the structure:

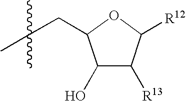

where R¹² is a purine or pyrimidine base, and R¹³ is hydrogen or —OH.

In some embodiments, the carotenoid analog or derivative may have the structures 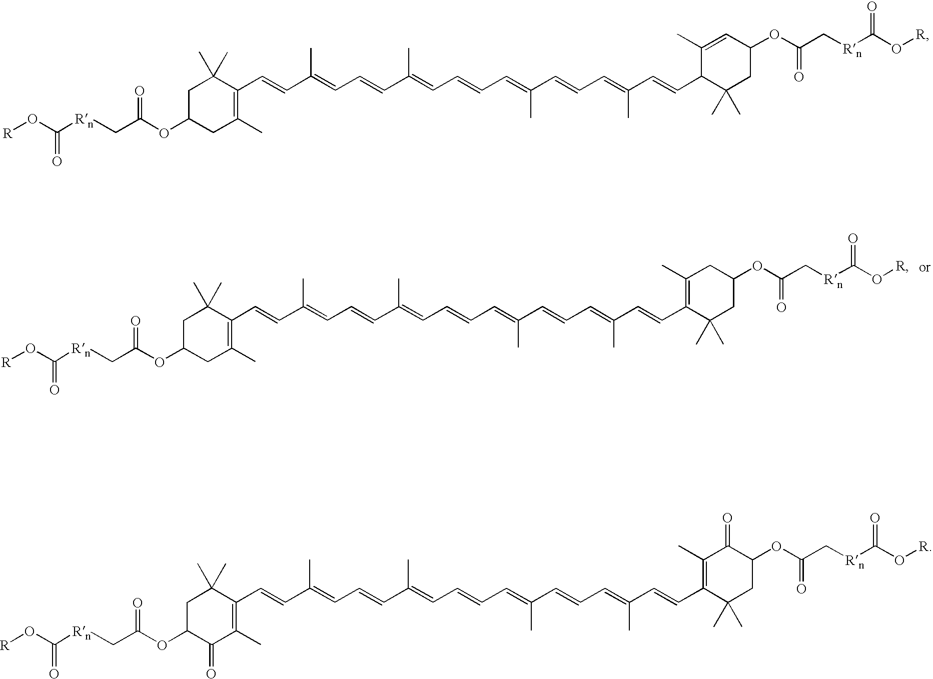
Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structures 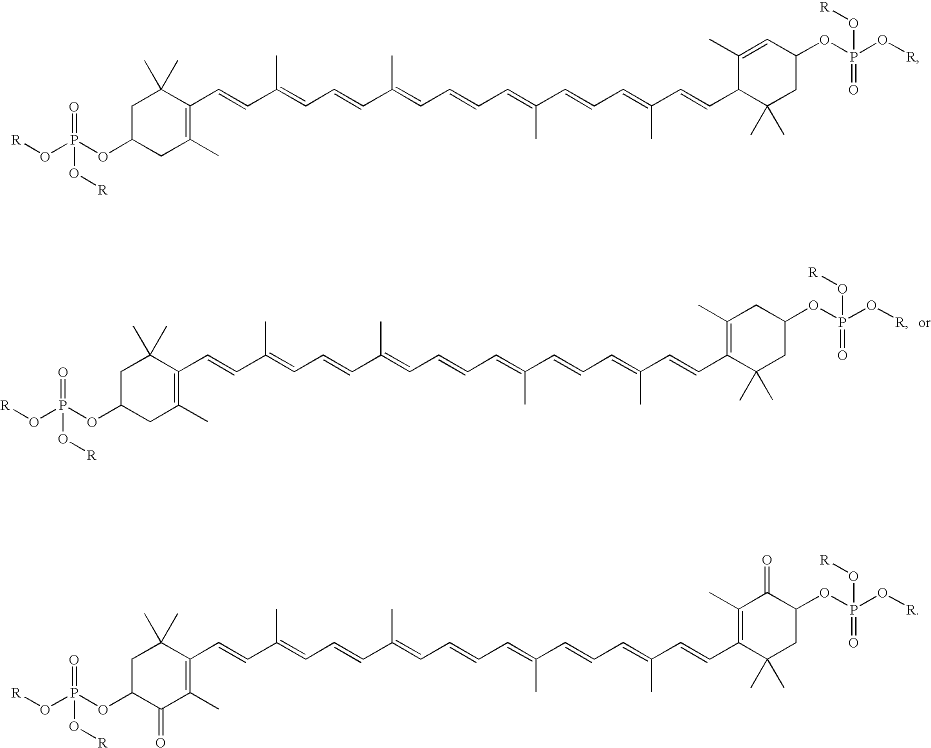
Each R may be independently H, alkyl, aryl, benzyl, Group IA metal (e.g., sodium), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. When R includes Vitamin C, Vitamin C analogs, or Vitamin C derivatives, some embodiments may include carotenoid analogs or derivatives having the structure 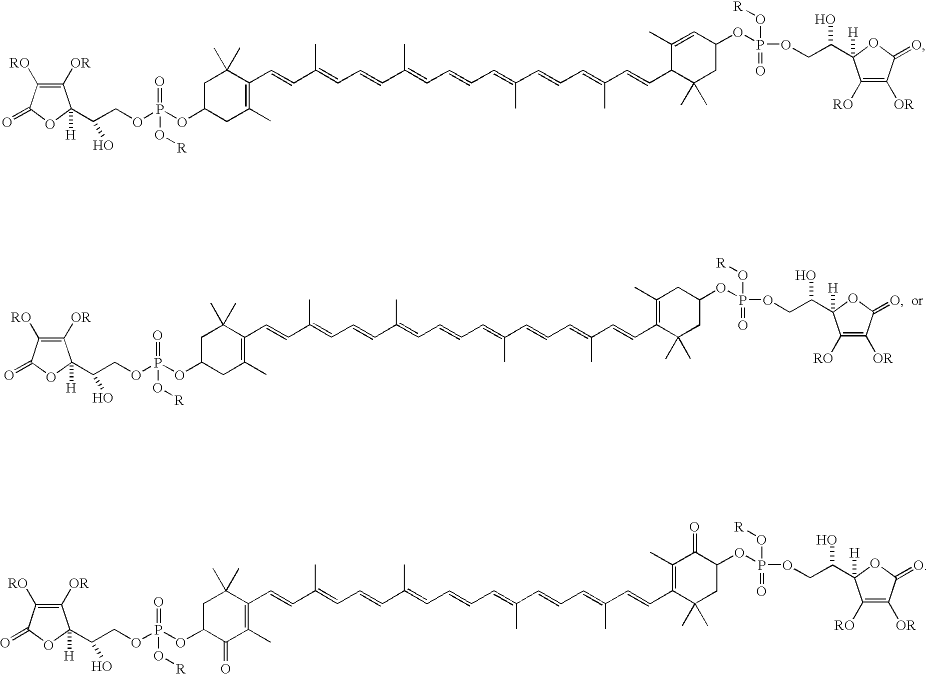
Each R may be independently H, alkyl, aryl, benzyl, or Group IA metal.

In some embodiments, the carotenoid derivative may have the structure:

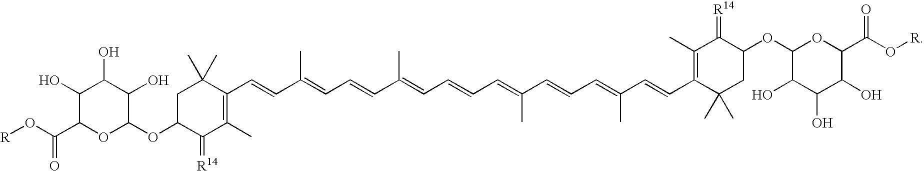

Each R¹⁴ may be independently O or H₂. Each R may be independently H, alkyl, benzyl, Group IA metal, co-antioxidant, or aryl.

Specific examples of carotenoid derivatives include, but are not limited to, the following compounds:

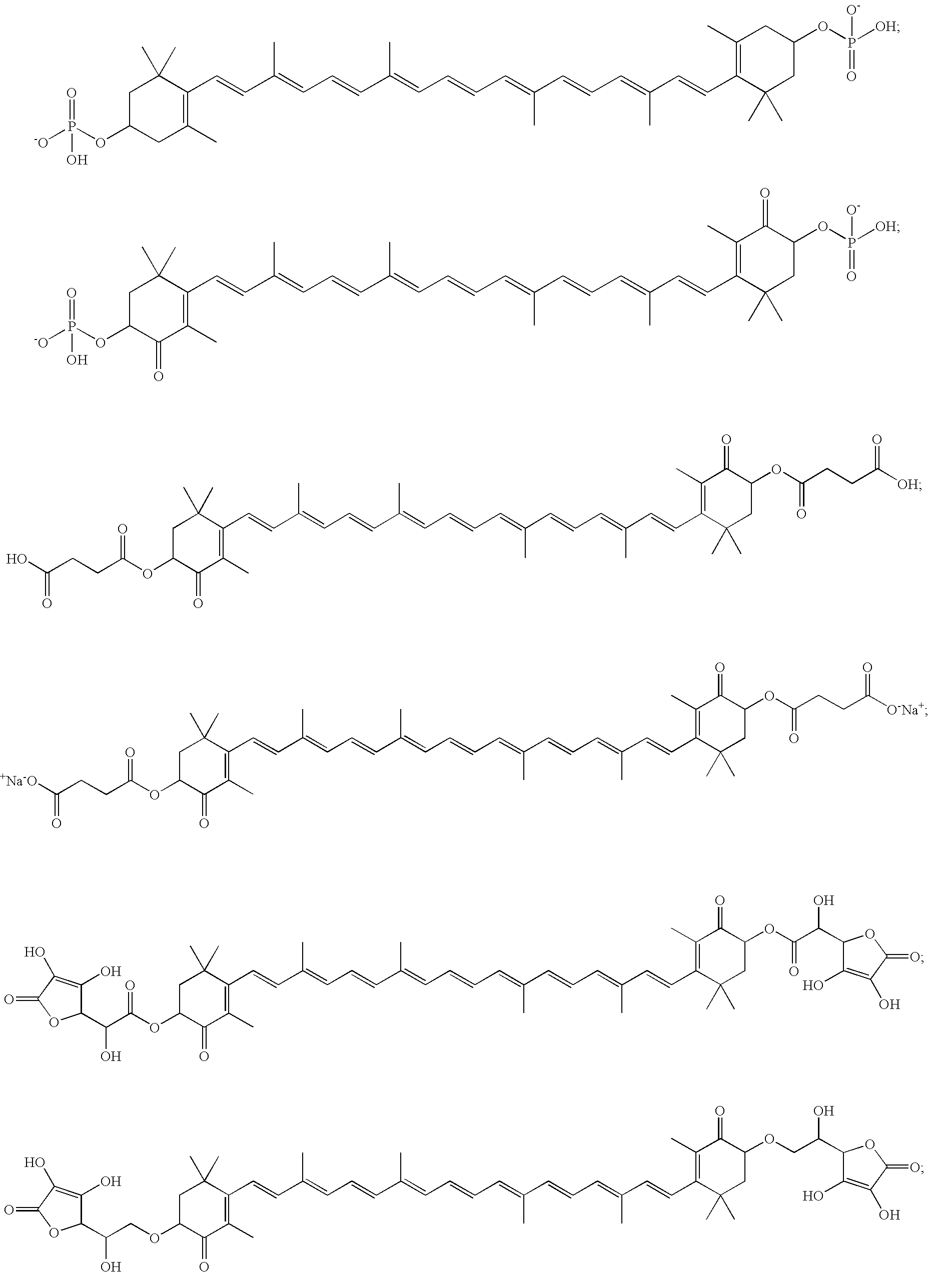

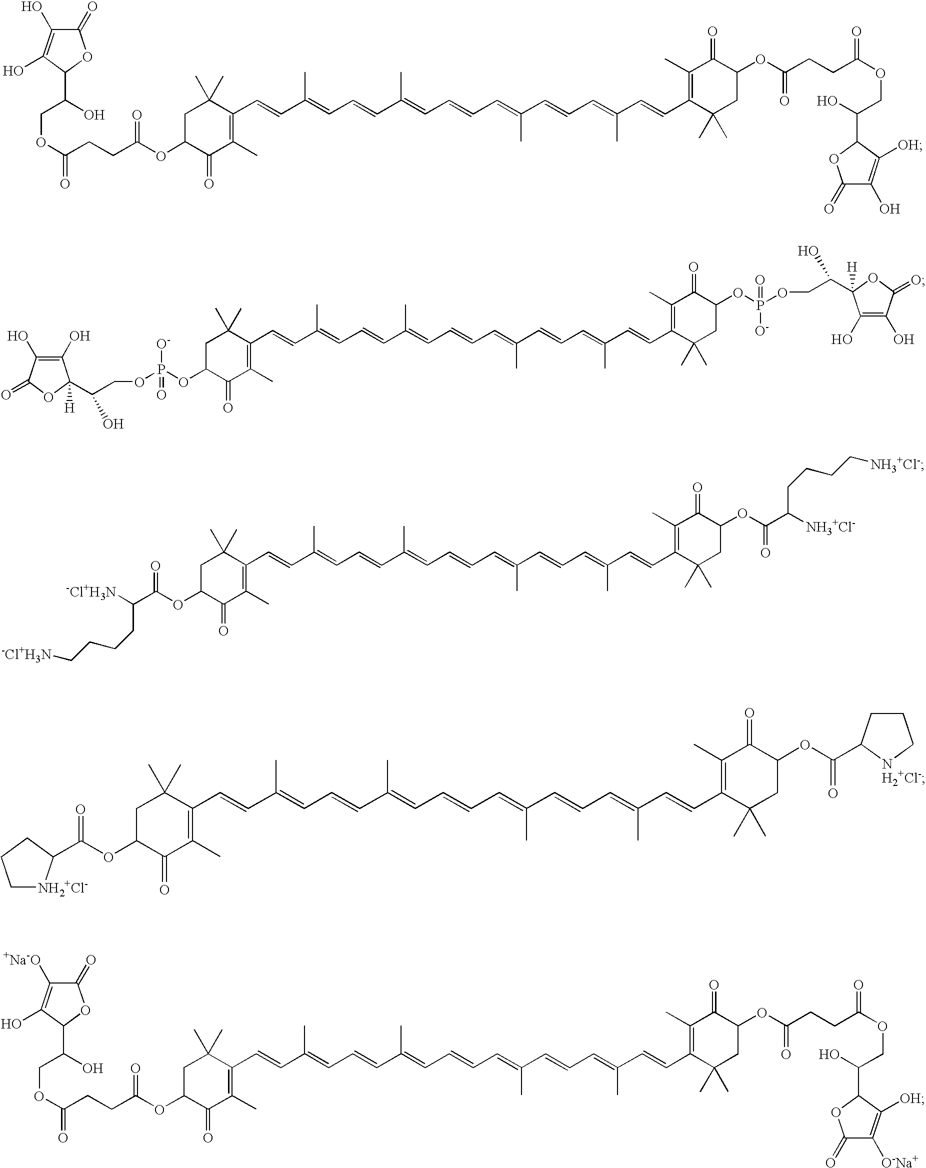

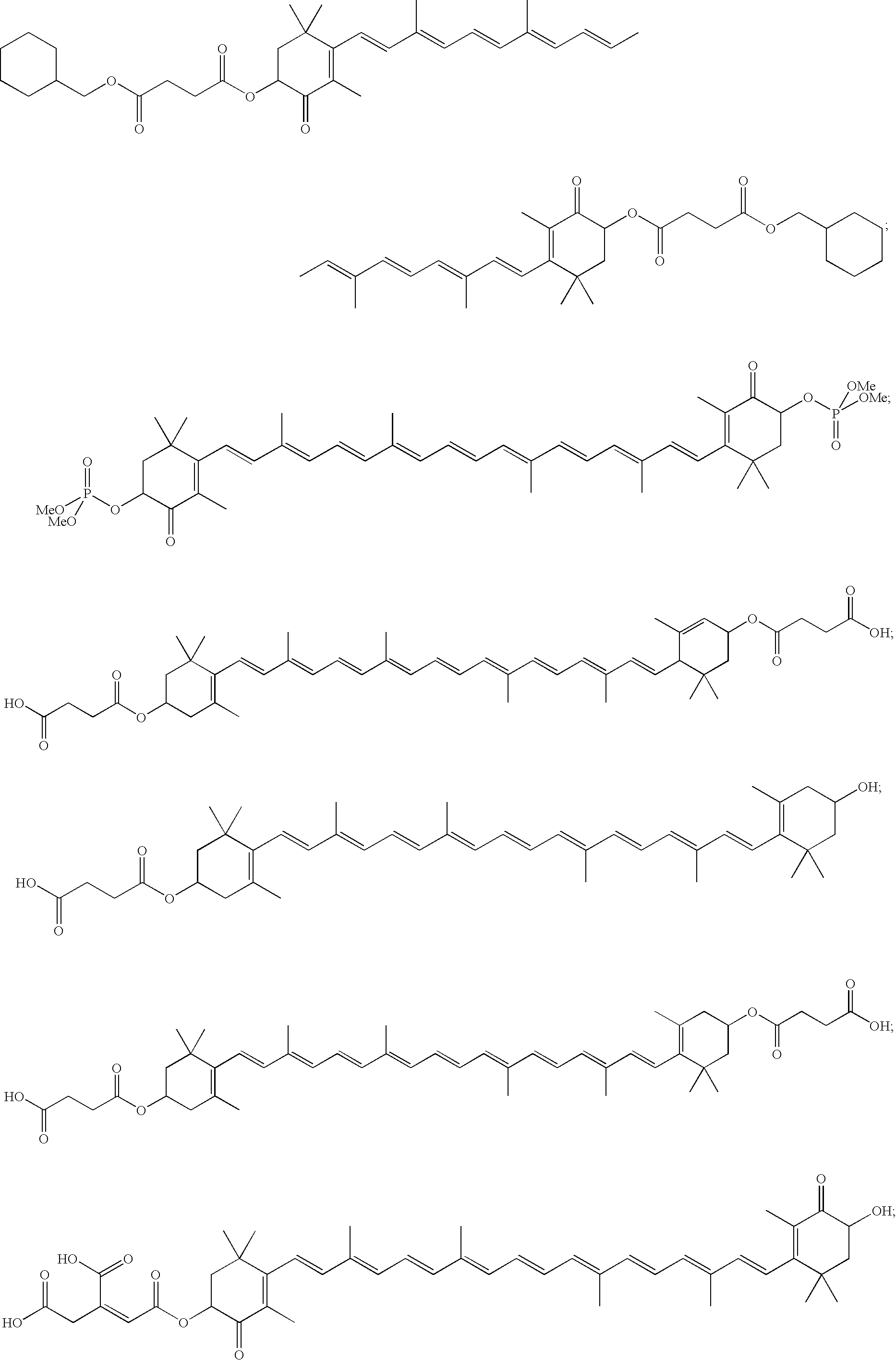

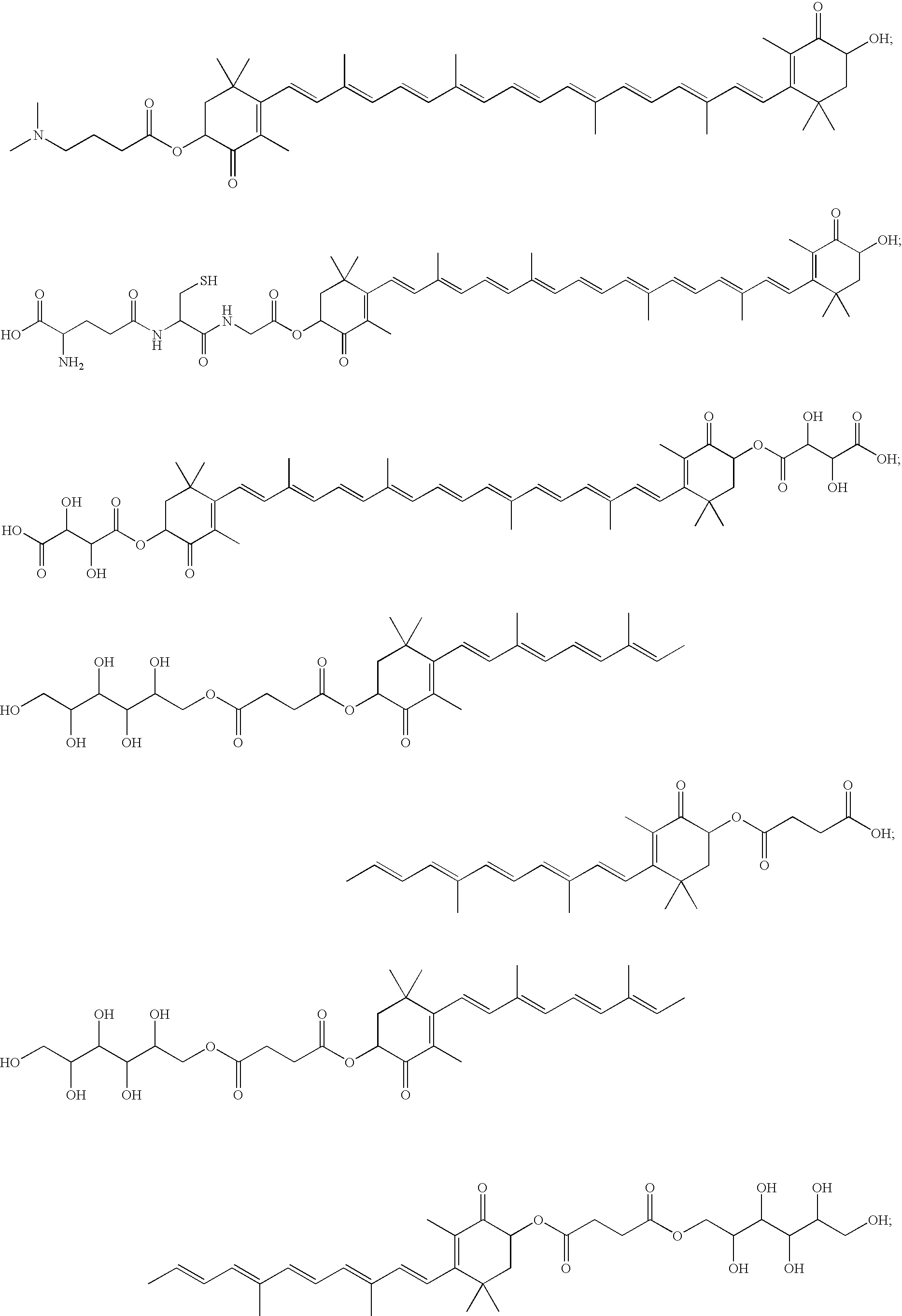

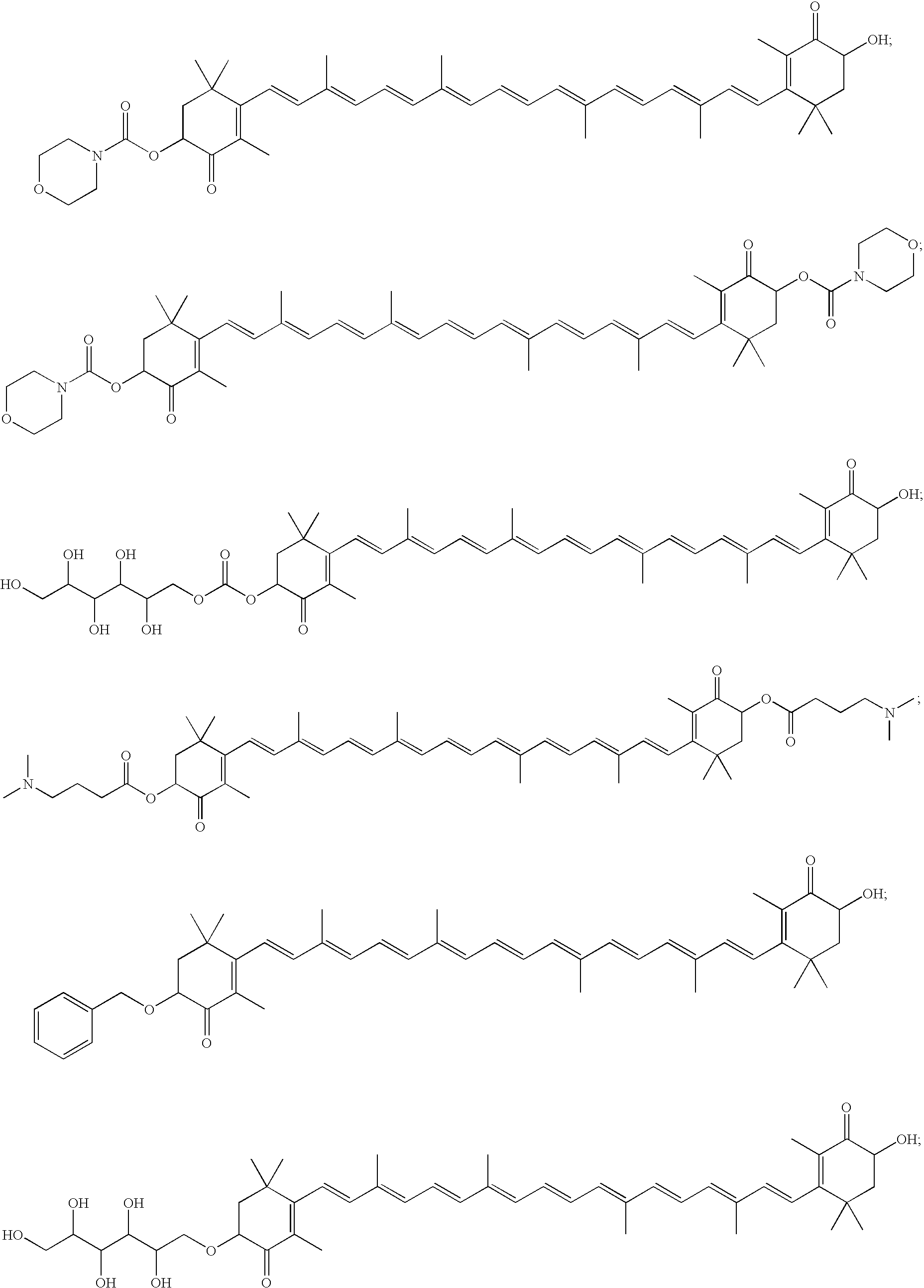

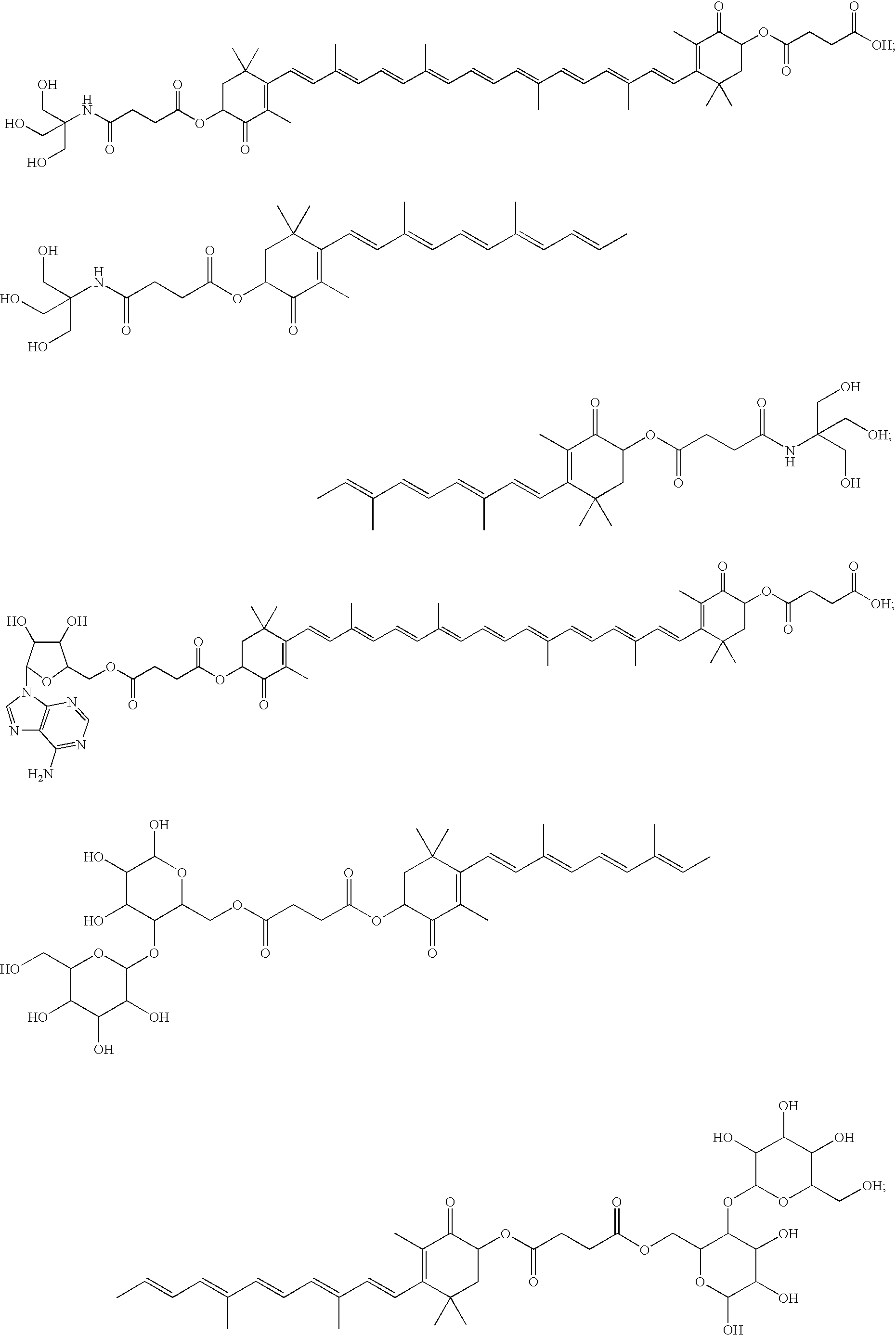

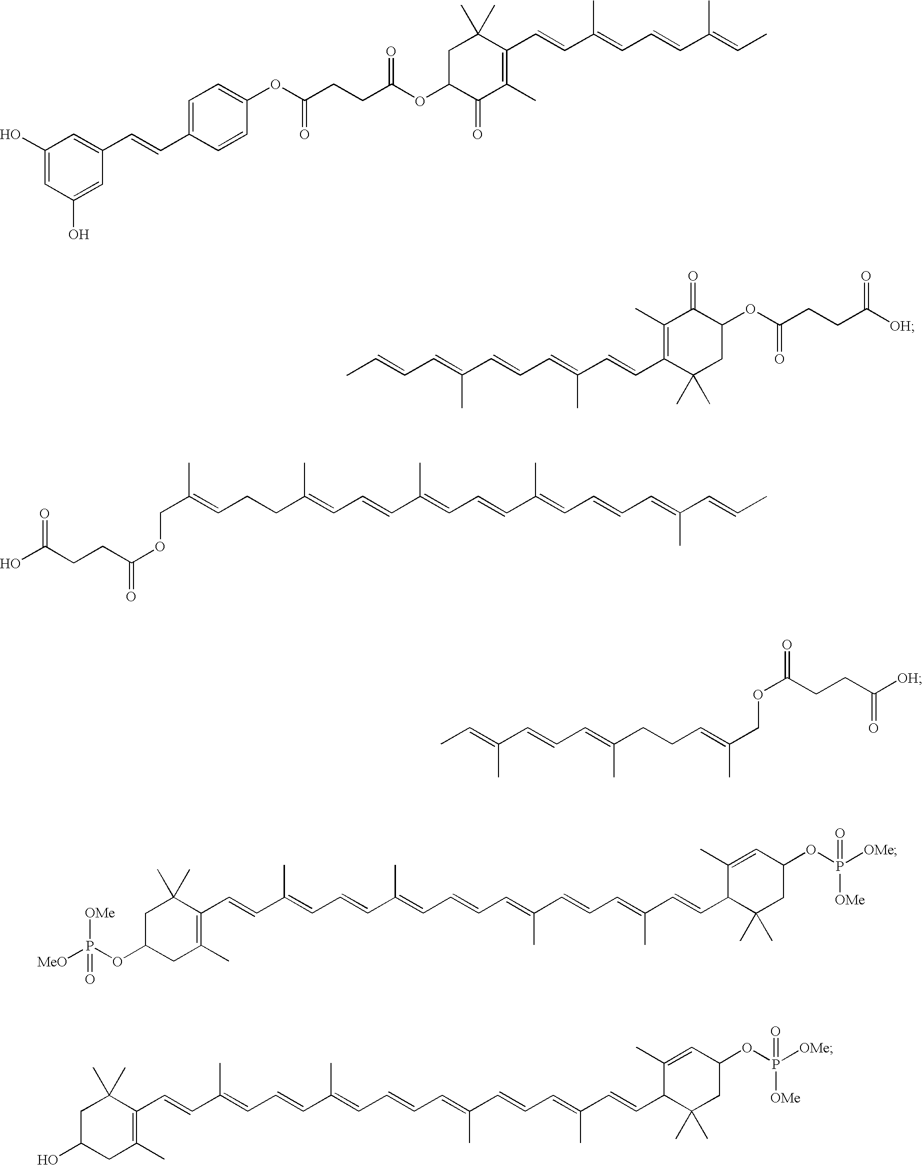

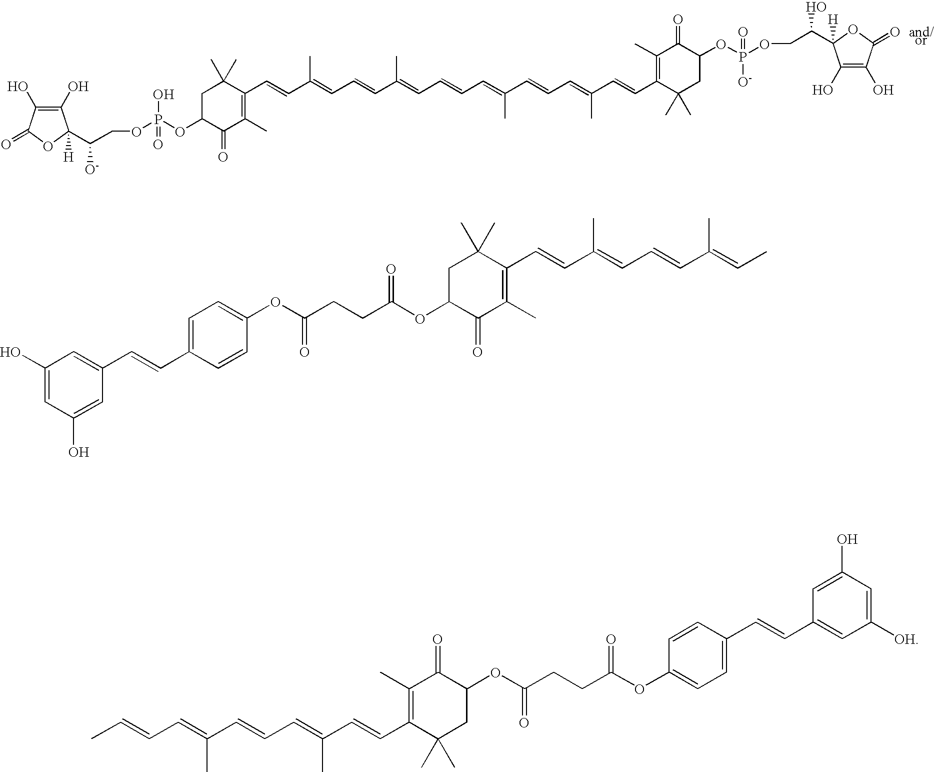

Further details regarding the synthesis of carotenoid derivatives and analogs is illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and U.S. patent application Ser. No. 11/106,378 filed on Apr. 14, 2005, entitled “CAROTENOID ANALOGS OR DERIVATIVES FOR THE INHIBITION AND AMELIORATION OF INFLAMMATION” to Lockwood et al. published on Nov. 24, 2005, as Publication No. U.S.-2005-0261254 both of which are incorporated by reference as though fully set forth herein.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/mL. In certain embodiments, water-soluble carotenoid analogs or derivatives may have a water solubility of greater than about 10 mg/mL. In certain embodiments, water-soluble carotenoid analogs or derivatives may have a water solubility of greater than about 20 mg/mL. In some embodiments, water-soluble carotenoid analogs or derivatives may have a water solubility of greater than about 50 mg/mL.

Naturally occurring carotenoids such as xanthophyll carotenoids of the C40 series, which includes commercially important compounds such as lutein, zeaxanthin, and astaxanthin, have poor aqueous solubility in the native state. Varying the chemical structure(s) of the esterified moieties may vastly increase the aqueous solubility and/or dispersibility of derivatized carotenoids.

In some embodiments, highly water-dispersible C40 carotenoid derivatives may include natural source RRR-lutein (β,ε-carotene-3,3′-diol) derivatives. Derivatives may be synthesized by esterification with inorganic phosphate and succinic acid, respectively, and subsequently converted to the sodium salts. Deep orange, evenly colored aqueous suspensions were obtained after addition of these derivatives to USP-purified water. Aqueous dispersibility of the disuccinate sodium salt of natural lutein was 2.85 mg/mL; the diphosphate salt demonstrated a >10-fold increase in dispersibility at 29.27 mg/mL. Aqueous suspensions may be obtained without the addition of heat, detergents, co-solvents, or other additives.

The direct aqueous superoxide scavenging abilities of these derivatives were subsequently evaluated by electron paramagnetic resonance (EPR) spectroscopy in a well-characterized in vitro isolated human neutrophil assay. The derivatives may be potent (millimolar concentration) and nearly identical aqueous-phase scavengers, demonstrating dose-dependent suppression of the superoxide anion signal (as detected by spin-trap adducts of DEPMPO) in the millimolar range. Evidence of card-pack aggregation was obtained for the diphosphate derivative with UV-Vis spectroscopy (discussed herein), whereas limited card-pack and/or head-to-tail aggregation was noted for the disuccinate derivative. Nadolski et al. 2006 is hereby incorporated by reference as though fully set forth herein. These lutein-based soft drugs may find utility in those commercial and clinical applications for which aqueous-phase singlet oxygen quenching and direct radical scavenging may be required.

The absolute size of a carotenoid derivative (in 3 dimensions) is important when considering its use in biological and/or medicinal applications. Some of the largest naturally occurring carotenoids are no greater than about C₅₀. This is probably due to size limits imposed on molecules requiring incorporation into and/or interaction with cellular membranes. Cellular membranes may be particularly co-evolved with molecules of a length of approximately 30 nm. In some embodiments, carotenoid derivatives may be greater than or less than about 30 nm in size. In certain embodiments, carotenoid derivatives may be able to change conformation and/or otherwise assume an appropriate shape, which effectively enables the carotenoid derivative to efficiently interact with a cellular membrane.

Although the above structure, and subsequent structures, depict alkenes in the E configuration this should not be seen as limiting. Compounds discussed herein may include embodiments where alkenes are in the Z configuration or include alkenes in a combination of Z and E configurations within the same molecule. The compounds depicted herein may naturally convert between the Z and E configuration and/or exist in equilibrium between the two configurations.

Compounds described herein embrace isomers mixtures, racemic, optically active, and optically inactive stereoisomers and compounds. Carotenoid analogs or derivatives may have increased water solubility and/or water dispersibility relative to some or all known naturally occurring carotenoids. In some embodiments, one or more co-antioxidants may be coupled to a carotenoid or carotenoid derivative or analog.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate, succinate, co-antioxidant (e.g., Vitamin C, Vitamin C analogs, Vitamin C derivatives, Vitamin E, Vitamin E analogs, Vitamin E derivatives, or flavonoids), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their bioavailability and hence proper evaluation of efficacy. Esterification or etherification may be useful to increase oral bioavailability, a fortuitous side effect of the esterification process, which can increase solubility in gastric mixed micelles. These compounds, upon introduction to the mammalian GI tract, are rapidly and effectively cleaved to the parent, non-esterified compounds, and enter the systemic circulation in that manner and form. The effect of the intact ester and/or ether compound on the therapeutic endpoint of interest can be obtained with parenteral administration of the compound(s). The net overall effect is an improvement in potential clinical utility for the lipophilic carotenoid compounds as therapeutic agents.

In one embodiment, a subject may be administered a pharmaceutical composition comprising a carotenoid analog or derivative. The analog or derivative may be broken down according to the following reaction:

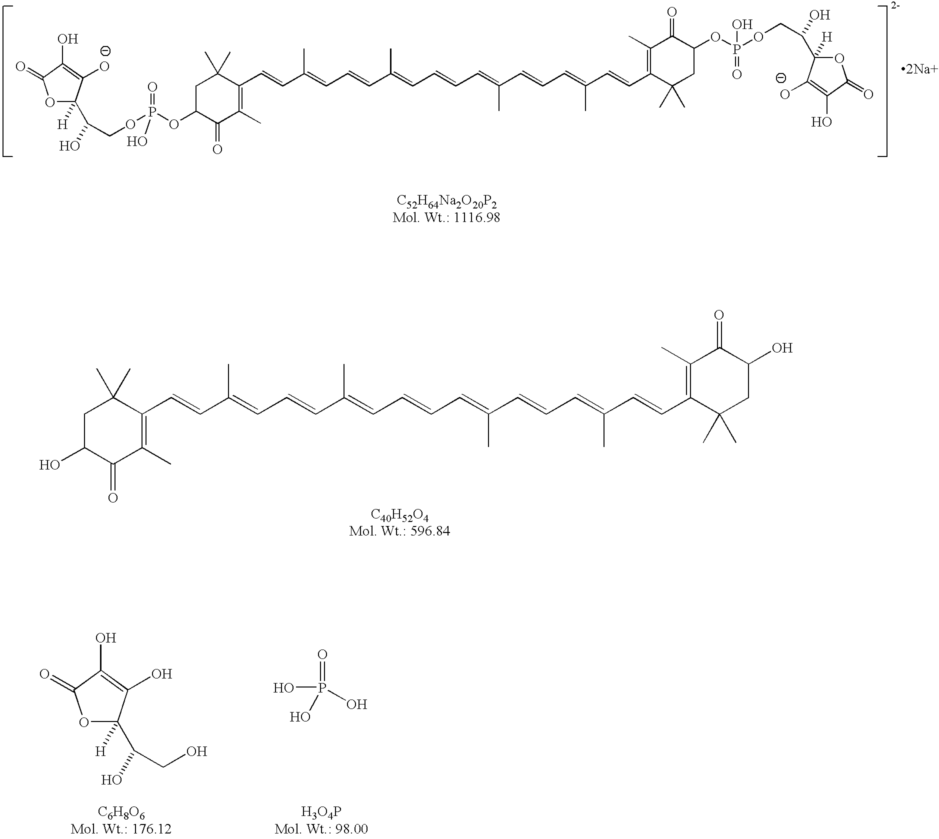

In some embodiments, the principles of retrometabolic drug design may be utilized to produce novel soft drugs from the asymmetric parent carotenoid scaffold (e.g., RRR-lutein (β,ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

Some embodiments may include solutions or pharmaceutical preparations of carotenoids and/or carotenoid derivatives combined with co-antioxidants, in particular vitamin C and/or vitamin C analogs or derivatives. Pharmaceutical preparations may include about a 2:1 ratio of vitamin C to carotenoid respectively.

In some embodiments, co-antioxidants (e.g., vitamin C) may increase solubility of the chemical compound. In certain embodiments, co-antioxidants (e.g., vitamin C) may decrease toxicity associated with at least some carotenoid analogs or derivatives. In certain embodiments, co-antioxidants (e.g., vitamin C) may increase the potency of the chemical compound synergistically. Co-antioxidants may be coupled (e.g., a covalent bond) to the carotenoid derivative. Co-antioxidants may be included as a part of a pharmaceutically acceptable formulation.

In some embodiments, more than one xanthophyll carotenoid or structural analog or derivative or synthetic intermediate of carotenoids may be synergistically combined. A composition may include one xanthophyll carotenoid or a structural carotenoid analog or derivative or synthetic intermediate combined with one or more different xanthophyll carotenoids or structural carotenoid analogs or derivatives or synthetic intermediates or co-antioxidants, either as derivatives or in solutions and/or formulations.

Certain embodiments may include administering a xanthophyll carotenoid or a structural carotenoid analogs or derivatives or synthetic intermediates alone or in combination to a subject such that at least a portion of the adverse effects of a visual disability are thereby reduced, inhibited and/or ameliorated. The xanthophyll carotenoid or a structural carotenoid analogs or derivatives or synthetic intermediates may be water-soluble and/or water dispersible derivatives. The carotenoid derivatives may include any substituent that substantially increases the water solubility of the naturally occurring carotenoid. The carotenoid derivatives may retain and/or improve the antioxidant properties of the parent carotenoid. The carotenoid derivatives may retain the non-toxic properties of the parent carotenoid. The carotenoid derivatives may have increased bioavailability, relative to the parent carotenoid, upon administration to a subject. The parent carotenoid may be naturally occurring.

Other embodiments may include the administering a composition comprised of the synergistic combination of more than one xanthophyll carotenoids or structural carotenoid analogs or derivatives or synthetic intermediates to a subject such that at least a portion of the adverse effects of a visual disability are thereby reduced, inhibited and/or ameliorated. The composition may be a “racemic” (i.e. mixture of the potential stereoisomeric forms) mixture of carotenoid derivatives. Included as well are pharmaceutical compositions comprised of structural analogs or derivatives or synthetic intermediates of carotenoids in combination with a pharmaceutically acceptable carrier. In one embodiment, a pharmaceutically acceptable carrier may be serum albumin. In one embodiment, structural analogs or derivatives or synthetic intermediates of carotenoids may be complexed with human serum protein such as, for example, human serum albumin (i.e., HSA) in a solvent. In an embodiment, HSA may act as a pharmaceutically acceptable carrier.

In some embodiments, a single stereoisomer of a structural analog or derivative or synthetic intermediate of carotenoids may be administered to a human subject in order to ameliorate a pathological condition. Administering a single stereoisomer of a particular compound (e.g., as part of a pharmaceutical composition) to a human subject may be advantageous (e.g., increasing the potency of the pharmaceutical composition). Administering a single stereoisomer may be advantageous due to the fact that only one isomer of potentially many may be biologically active enough to have the desired effect.

In some embodiments, compounds described herein may be administered in the form of nutraceuticals. Generally a nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingredients, and a stimulant functional food or pharmafood. Within the context of the description herein where the composition, use and/or delivery of pharmaceuticals are described nutraceuticals may also be composed, used, and/or delivered in a similar manner where appropriate.

Dosage and Administration

The xanthophyll carotenoid, carotenoid derivative or analog may be administered at a dosage level up to conventional dosage levels for xanthophyll carotenoids, carotenoid derivatives or analogs, but will typically be less than about 2 gm per day. Suitable dosage levels may depend upon the overall systemic effect of the chosen xanthophyll carotenoids, carotenoid derivatives or analogs, but typically suitable levels will be about 0.001 to 50 mg/kg body weight of the patient per day, from about 0.005 to 30 mg/kg per day, or from about 0.05 to 10 mg/kg per day. The compound may be administered on a regimen of up to 6 times per day, between about 1 to 4 times per day, or once per day.

In the case where an oral composition is employed, a suitable dosage range is, e.g. from about 0.01 mg to about 100 mg of a xanthophyll carotenoid, carotenoid derivative or analog per kg of body weight per day, preferably from about 0.1 mg to about 10 mg per kg and for cytoprotective use from 0.1 mg to about 100 mg of a xanthophyll carotenoid, carotenoid derivative or analog per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the structural carotenoid analog. Liposomal formulations, in which mixtures of the structural carotenoid analog or derivative with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress or the development of a visual disability.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

### EXAMPLES

Having now described the invention, the same will be more readily understood through reference to the following example(s), which are provided by way of illustration, and are not intended to be limiting of the present invention.

General.

Natural source lutein (90%) was obtained from ChemPacific, Inc. (Baltimore, Md.) as a red-orange solid and was purified by dissolving in a minimal volume of CH₂Cl₂, passed through a 0.45 μm filter, and concentrated in vacuo. This process was repeated three times. All other reagents and solvents used were purchased from Acros (New Jersey, USA) and were used without further purification. All reactions were performed under N₂ atmosphere. All flash chromatographic purifications were performed on Natland International Corporation 230-400 mesh silica gel using the indicated solvents. LC/MS (APCI) was recorded on an Agilent 1100 LC/MSD VL system; column: Zorbax Eclipse XDB-C18 Rapid Resolution (4.6×75 mm, 3.5 μm); temperature: 25° C.; starting pressure: 105 bar; flow rate: 1.0 mL/min; mobile phase (% A=0.025% trifluoroacetic acid in H₂O, % B=0.025% trifluoroacetic acid in acetonitrile). Gradient program: 70% A/30% B (start), step gradient to 50% B over 5 min, step gradient to 98% B over 8.30 min, hold at 98% B over 25.20 min, step gradient to 30% B over 25.40 min; PDA Detector: 470 nm. The presence of trifluoroacetic acid in the LC eluents acts to protonate lutein disuccinate and diphosphate salts to give the free diacid forms, yielding M+=768 for the disuccinate salt and M+=728 for the diphosphate salt. LRMS: +mode; APCI: atmospheric pressure chemical ionization, ion collection using quadrapole.

Lutein (β,ε-carotene-3,3′-diol) (1). LC/MS (APCI): 9.95 min (2.78%), λₘₐₓ 423 nm (70%), 446 nm (100%), 474 nm (80%); 10.58 min (3.03%), λₘₐₓ 423 nm (73%), 446 nm (100%), 474 nm (82%); 11.10 min (4.17%), λₘₐₓ 423 nm (68%), 447 nm (100%), 474 nm (79%); 12.41 min (90.02%), λₘₐₓ 423 nm (73%), 447 nm (100%), 474 nm (83%); m/z 568 M⁺(69%), 551 [M_H2O+H]⁺(100%), 533 (8%).

β,ε-Carotenyl 3,3′-disuccinate (2). To a solution of lutein (1) (0.50 g, 0.879 mmol) in CH₂Cl₂ (8 mL) were added N,N-diisopropylethylamine (3.1 mL, 17.58 mmol) and succinic anhydride (0.88 g, 8.79 mmol). The solution was stirred at room temperature overnight and then diluted with CH₂Cl₂ and quenched with cold 5% aqueous HCl. The aqueous layer was extracted two times with CH₂Cl₂ and the combined organic layer was washed three times with 5% aqueous HCl, dried over Na₂SO₄, and concentrated to yield disuccinate 2 (0.433 g, 64%) as a red-orange solid; LC/MS (APCI):10.37 min (8.42%), λₘₐₓ 423 nm (74%), 446 nm (100%), 474 nm (83%); m/z 769 [M+H]⁺(8%), 668 [M_C4O3H4]⁺(7%), 650 (100%), 532 (22%); 11.78 min (90.40%), λₘₐₓ 269 nm (18%), 423 nm (68%), 446 nm (100%), 474 nm (80%); m/z 769 [M+H]⁺(7%), 668 [M-C4O3H4]⁺(9%), 650 (100%), 532 (23%).

β,ε-Carotenyl 3,3′-disuccinate sodium salt (3). To a solution of disuccinate 2 (0.32 g, 0.416 mmol) in CH₂Cl₂ (6 mL) at 0° C. was added dropwise sodium methoxide (25% wt in methanol; 0.20 mL, 0.874 mmol). The solution was diluted with water, and the clear, red-orange aqueous solution was lyophilized to yield 3 (0.278 g, 91%) as a red-orange, hygroscopic solid; LC/MS (APCI): 11.75 min (91.32%), λₘₐₓ 269 nm (16%), 423 nm (70%), 446 nm (100%), 474 nm (82%); m/z 769 [M+H]⁺(7%), 668 [M-C4O3H4]⁺(9%), 650 (100%), 532 (23%).

Tribenzyl phosphite (4). To a well-stirred solution of phosphorus trichloride (1.7 mL, 19.4 mmol) in Et₂O (430 mL) at 0° C. was added dropwise a solution of triethylamine (8.4 mL, 60.3 mmol) in Et₂O (20 mL), followed by a solution of benzyl alcohol (8.1 mL, 77.8 mmol) in Et₂O (20 mL). The mixture was stirred at 0° C. for 30 min and then at room temperature overnight. The mixture was filtered and the filtrate concentrated to give a colorless oil. Silica chromatography (hexanes/Et₂O/triethylamine, 4/1/1%) of the crude product yielded 4 (5.68 g, 83%) as a clear, colorless oil that was stored under N₂ at −20° C.; ¹H NMR: δ 7.38 (15H, m), 4.90 (6H, d).

Dibenzyl phosphoroiodidate (5). To a solution of tribenzyl phosphite (5.43 g, 15.4 mmol) in CH₂Cl₂ (8 mL) at 0° C. was added I₂ (3.76 g, 14.8 mmol). The mixture was stirred at 0° C. for 10 min or until the solution became clear and colorless. The solution was then stirred at room temperature for 10 min and used directly in the next step.

(Monobenzyl-phosphoryloxy)-(phosphoryloxy)-β,ε-carotene (6). To a solution of lutein (1) (0.842 g, 1.48 mmol) in CH₂Cl₂ (8 mL) were added pyridine (4.8 mL, 59.2 mmol). The solution was stirred at 0° C. for 5 min and then freshly prepared 5 (14.8 mmol) in CH₂Cl₂ (8 mL) was added dropwise to the mixture at 0° C. The solution was stirred at 0° C. for 1 h and then diluted with CH₂Cl₂ and quenched with brine. The aqueous layer was extracted twice with CH₂Cl₂ and the combined organic layer was washed once with NaSSO₄, once with brine, then dried over Na₂SO₄ and concentrated. Pyridine was removed by azeotropic distillation using toluene to yield mono benzylprotected diphosphate 6, used in the next step without further purification; LC/MS (APCI): 9.67 min (13.31%), λₘₐₓ 268 nm (26%), 423 nm (74%), 446 nm (100%), 476 nm (84%); m/z 850 (5%), 825 (4%), 810 (100%), 532 (96%); 10.02 min (86.69%), λₘₐₓ 268 nm (26%), 423 nm (72%), 446 nm (100%), 476 nm (89%); m/z 850 (5%), 825 (4%), 810 (100%), 532 (92%).

3,3′-Diphosphoryloxy-β,ε-carotene (7). To a solution of 6 (1.48 mmol) in CH₂Cl₂ (10 mL) at 0° C. were added pyridine (1.2 mL, 14.8 mmol) and then bromotriethylsilane (0.97 mL, 7.40 mmol). The solution was stirred at 0° C. for 30 min, quenched with triethylamine, diluted with CH₂Cl₂, and then concentrated to yield crude diphosphate 7 as a red-orange oil, used in the next step without further purification; LC/MS (APCI): 8.90 min (54.88%), λₘₐₓ 268 nm (20%), 423 nm (70%), 446 nm (100%), 476 nm (90%); m/z 693 (5%), 639 (48%), 555 (42%), 538 (100%); 9.18 min (43.33%), λₘₐₓ 423 nm (78%), 446 nm (100%), 476 nm (91%); m/z 693 (7%), 639 (45%), 555 (38%), 538 (100%).

3,3′-Diphosphoryloxy-β,ε-carotene sodium salt (8). To a solution of crude 7 (1.48 mmol) in CH₂Cl₂ (10 mL) at 0° C. was added dropwise sodium methoxide (25% wt in methanol; 6.77 mL, 29.6 mmol). The solution was stirred at room temperature overnight and then diethyl ether was added to the salt. The suspension was centrifuged and the supernatant discarded. Water was added to the salt and the suspension was centrifuged and the supernatant discarded. The salt was redissolved in methanol and diluted with water. Lyophilization of the clear, red-orange aqueous solution yielded 8 (0.38 g, 35% over three steps) as a red-orange, hygroscopic solid; LC/MS (APCI): 8.54 min (25.86%), λₘₐₓ 268 nm (25%), 423 nm (74%), 446 nm (100%), 474 nm (68%); 8.85 min (31.13%), λₘₐₓ 268 nm (20%), 423 nm (66%), 446 nm (100%), 474 nm (80%), m/z 912 (50%), 780 (18%), 692 (7%), 630 (100%), 550 (45%);9.15 min (30.62%), λₘₐₓ 268 nm (23%), 423 nm (75%), 446 nm (100%), 474 nm (86%), m/z 912 (41%), 780 (15%), 692 (5%), 630 (100%), 550 (43%); 9.45 min (12.40%), λₘₐₓ 268 nm (21%), 335 nm (16%), 423 nm (76%), 446 nm (100%), 474 nm (80%).

UV/Visible Spectroscopy.

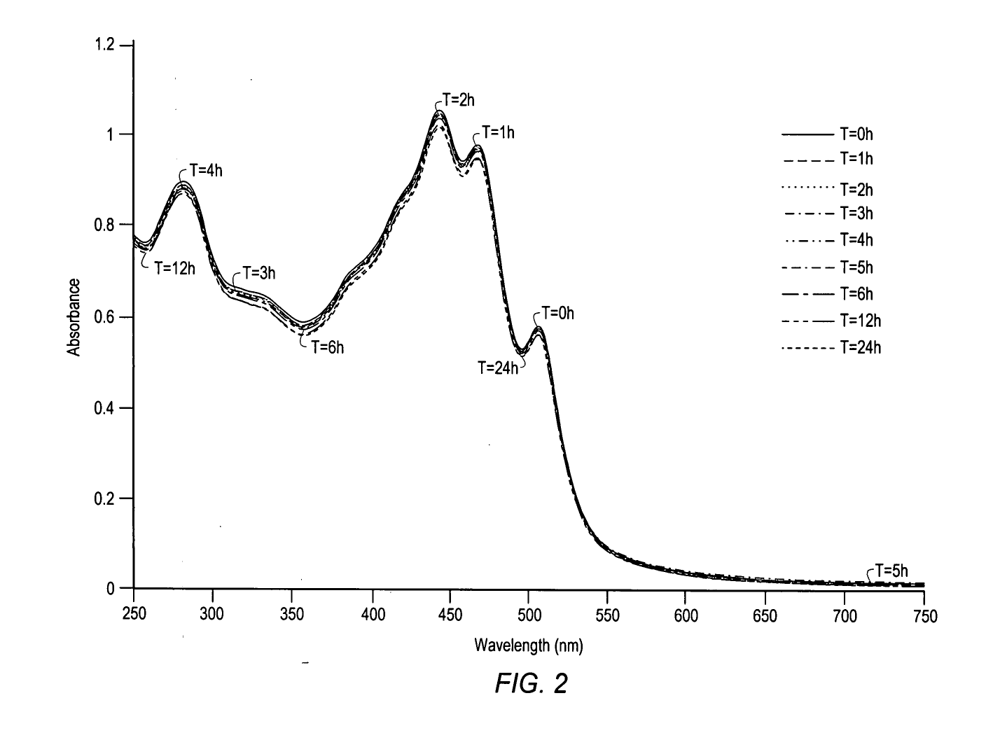

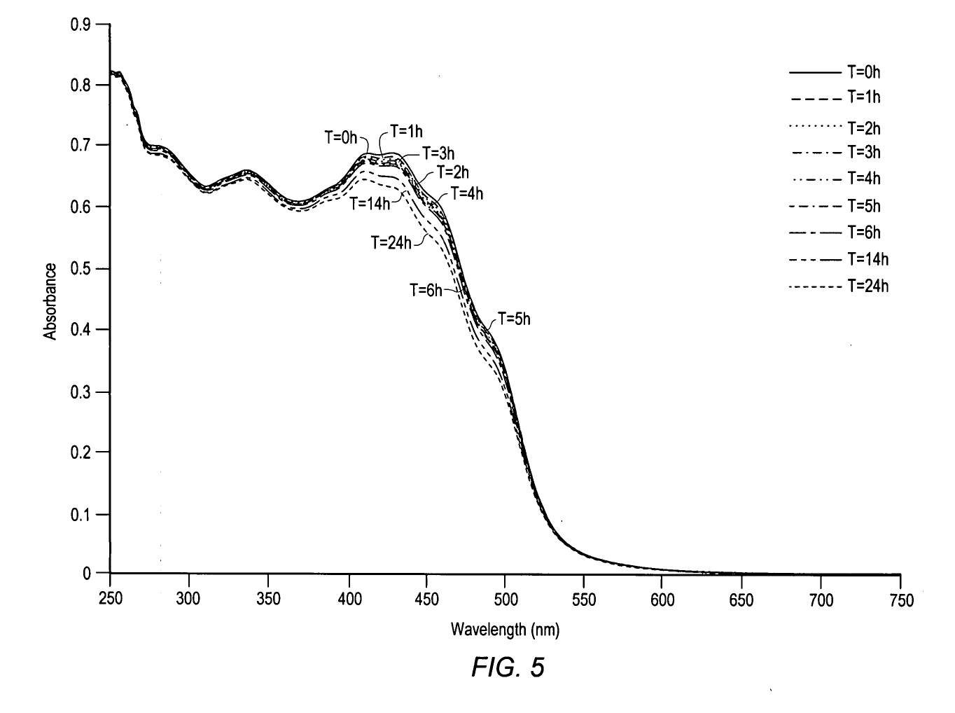

For spectroscopic sample preparations, 3 and 9 were dissolved in the appropriate solvent to yield final concentrations of approximately 0.01 mM and 0.2 mM, respectively. The solutions were then added to a rectangular cuvette with 1 cm path length fitted with a glass stopper. The absorption spectrum was subsequently registered between 250 and 750 nm. All spectra were accumulated one time with a bandwidth of 1.0 nm at a scan speed of 370 nm/min. For the aggregation time-series measurements, spectra were obtained at baseline (immediately after solvation; time zero) and then at the same intervals up to and including 24 hours post-solvation (see FIG. 2-FIG. 7). Concentration was held constant in the ethanolic titration of the diphosphate lutein sodium salt, for which evidence of card-pack aggregation was obtained (FIG. 5-FIG. 7).

Determination of Aqueous Solubility/Dispersibility.

30.13 mg of 3 was added to 1 mL USP-purified water. The sample was rotated for 2 h and then centrifuged for 5 min. After centrifuging, solid was visible in the bottom of the tube. A 125 μL aliquot of the solution was then diluted to 25 mL. The sample was analyzed by UV/Vis spectroscopy at 436 nm, and the absorbance was compared to a standard curve compiled from four standards of known concentration. The concentration of the original supernatant was calculated to be 2.85 mg/mL and the absorptivity was 36.94 AU mL/cm mg. Slight error may have been introduced by the small size of the original aliquot.

Next, 30.80 mg of 8 was added to 1 mL USP-purified water. The sample was rotated for 2 h and then centrifuged for 5 min. After centrifuging, solid was visible in the bottom of the tube. A 125 μL aliquot of the solution was then diluted to 25 mL. The sample was analyzed by UV/Vis spectroscopy at 411 nm, and the absorbance was compared to a standard curve compiled from four standards of known concentration. The concentration of the original supernatant was calculated to be 29.27 mg/mL and the absorptivity was 2.90 AU mL/cm mg. Slight error may have been introduced by the small size of the original aliquot.

Leukocyte Isolation and Preparation.

Human polymorphonuclear leukocytes (PMNs) were isolated from freshly sampled venous blood of a single volunteer (S.F.L.) by Percoll density gradient centrifugation as described previously. Briefly, each 10 mL of whole blood was mixed with 0.8 mL of 0.1 M EDTA and 25 mL saline. The diluted blood was then layered over 9 mL Percoll at a specific density of 1.080 g/mL. After centrifugation at 400 g for 20 min at 20° C., the plasma, mononuclear cell, and Percoll layers were removed. Erythrocytes were subsequently lysed by addition of 18 mL ice-cold water for 30 s, followed by 2 mL of 10× PIPES buffer (25 mM PIPES, 110 mM NaCl, and 5 mM KCl, titrated to pH 7.4 with NaOH). Cells were then pelleted at 4° C., the supernatant was decanted, and the procedure was repeated. After the second hypotonic cell lysis, cells were washed twice with PAG buffer [PIPES buffer containing 0.003% human serum albumin (HSA) and 0.1% glucose]. Afterward, PMNs were counted by light microscopy on a hemocytometer. The isolation yielded PMNs with a purity of >95%. The final pellet was then suspended in PAG-CM buffer (PAG buffer with 1 mM CaCl₂ and 1 mM MgCl₂).

EPR Measurements.

All EPR measurements were performed using a Bruker ER 300 EPR spectrometer operating at X-band with a TM₁₁₀ cavity as previously described. The microwave frequency was measured with a Model 575 microwave counter (EIP Microwave, Inc., San Jose, Calif.). To measure superoxide anion (O₂⁻) generation from phorbol-ester (PMA)-stimulated PMNs, EPR spin-trapping studies were performed using the spintrap DEPMPO (Oxis, Portland, Oreg.) at 10 mM. 1×10⁶ PMNs were stimulated with PMA (1 ng/mL) and loaded into capillary tubes for EPR measurements. To determine the radical scavenging ability of 3 and 8 in aqueous and ethanolic formulations, PMNs were pre-incubated for 5 min with test compound, followed by PMA stimulation.

Instrument settings used in the spin-trapping experiments were as follows: modulation amplitude, 0.32 G; time constant, 0.16 s; scan time, 60 s; modulation frequency, 100 kHz; microwave power, 20 mW; and microwave frequency, 9.76 GHz. The samples were placed in a quartz EPR .at cell, and spectra were recorded. The component signals in the spectra were identified and quantified as reported previously.

Synthesis

Schemes 1 and 2 depict the syntheses of two water-dispersible lutein derivatives, the sodium salts of lutein disuccinate and lutein diphosphate, as seen in Schemes 1 and 2. Derivatizing hydrophobic carotenoids may impart water-dispersibility.

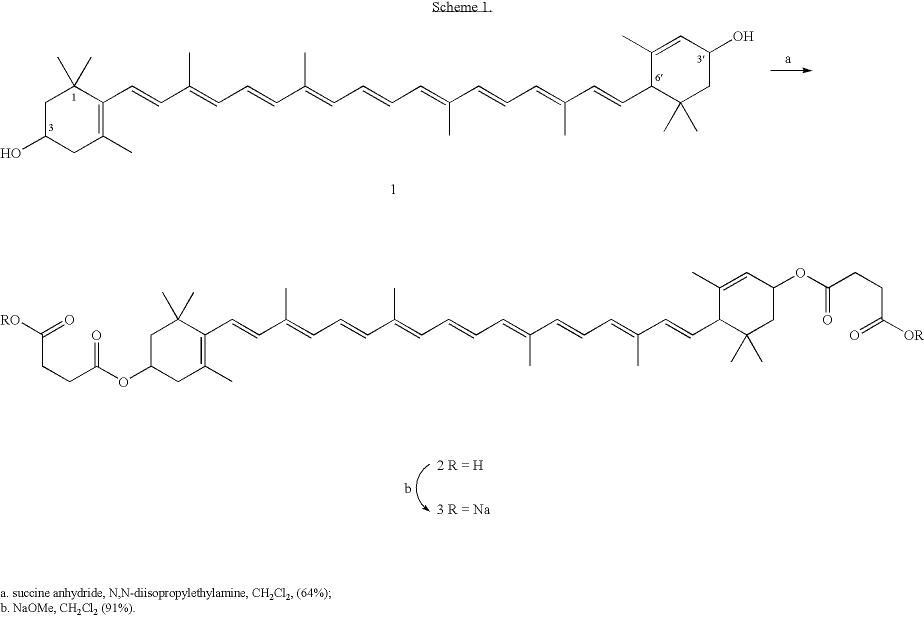

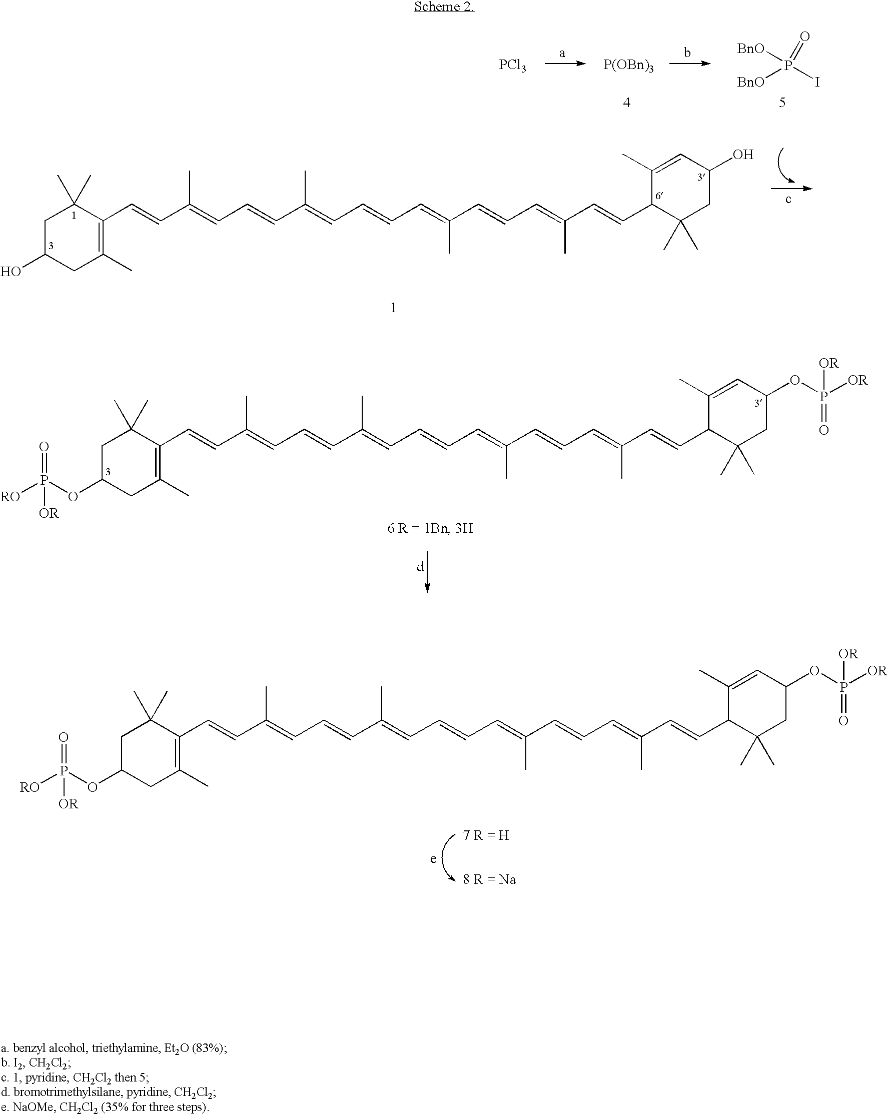

As seen in Scheme 1, the synthesis of disuccinate salt 3 began with succinylation of natural source lutein using succinic anhydride and Hünig base. Disuccinylation of lutein was optimized by running the reaction in a concentrated fashion and using modest excesses of anhydride and base. Aqueous acidic workup yielded disuccinate 2, such that excess reagents and reaction byproducts were removed by copiously extracting the organic layer with dilute HCl. The resulting viscous, red-orange oil was washed or slurried with hexanes to remove non-polar impurities. The water-dispersible derivative (3) was generated by treating 2 with methanolic sodium methoxide. The reaction was quenched with water and the resulting red-orange aqueous layer was first extracted with Et₂O, then lyophilized to provide the sodium salt in good yield.

Successful diphosphorylation of lutein may be achieved using dimethyl phosphoroiodidate, formed in situ by reacting commercially available trimethyl phosphite with iodine. A certain degree of success in removing all four diphosphate methyl groups may be realized when using bromotrimethylsilane in the presence of N,O-bis(trimethylsilyl)acetamide. However, this deprotection protocol may not be optimal in that methyl group dealkylation was usually accompanied by the significant decomposition of lutein phosphate.

A three-step method to provide the sodium salt of lutein diphosphate (9) may be achieved using benzyl esters as protecting groups for the lutein phosphoric acids (Scheme 2). Natural source lutein may be phosphorylated using dibenzyl phosphoroiodidate, formed in situ by reacting tribenzyl phosphite with iodine. As seen in Scheme 2, tribenzyl phosphite may be prepared by the addition of benzyl alcohol to phosphorus trichloride in the presence of triethylamine. Silica gel chromatography of the crude reaction mixture yielded tribenzyl phosphite in good yield. Compound 6 was formed by treating natural source lutein with freshly prepared dibenzyl phosphoroiodidate in the presence of pyridine. Aqueous workup of the reaction followed by the removal of pyridine by azeotropic distillation using toluene provided a crude red oil contaminated with excess reagents and reaction byproducts. Non-polar impurities were then removed from the crude product mixture by alternately washing or slurrying with hexanes and Et₂O to give 6 as confirmed by LC/MS analysis.

Advantageous dealkylation of two of the four benzyl esters of the phosphoric acid moieties occurred during the phosphorylation reaction, presumably at the more sensitive allylic 3′ phosphate. As seen in Scheme 2, the attempted removal of the phosphoric acid benzyl esters of 6 using LiOH—H₂O resulted in the generation of a less polar product versus compound 6, exhibiting a molecular ion of 828 as noted by LC/MS analysis. Under these reaction conditions, it is believed that dephosphorylation at one of the two hydroxyls of the lutein derivative occurred rather than the desired debenzylation to give compound 7. Such data indirectly support compound 6's structure and thus the occurrence of bis-dealkylation at one phosphate versus mono-dealkylation at both phosphates as an additional result of the phosphorylation of lutein. If mono-dealkylation at both phosphates occurred during phosphorylation, then treatment of the resulting product with LiOH—H₂O would have produced a lutein derivative possessing one phosphoric acid containing only one benzyl ester, exhibiting a molecular ion of 738 upon LC/MS analysis.

Following Scheme 2, successful dealkylation of the phosphate protecting groups of 6 may be achieved using bromotrimethylsilane in the presence of N,O-bis(trimethylsilyl)acetamide. A significant amount of excess reagents and reaction byproducts were removed from the resulting red oil by alternately washing or slurrying the crude mixture with ethyl acetate and CH₂Cl₂ to provide diphosphate 8 as an orange oil. As seen in Scheme 2, the sodium salt of lutein diphosphate (9) was generated by treating 8 with methanolic sodium methoxide. The resulting crude orange solid was washed or slurried with methanol and then dissolved in water. The aqueous layer was extracted first with CH₂Cl₂, then with ethyl acetate, and again with CH₂Cl₂. Lyophilization of the red-orange aqueous solution gave the sodium salt as an orange, hygroscopic solid. The phosphorylation process provided the desired water-dispersible lutein derivative 9 in good yield over the three steps. UV/Vis spectral properties in organic and aqueous solvents.

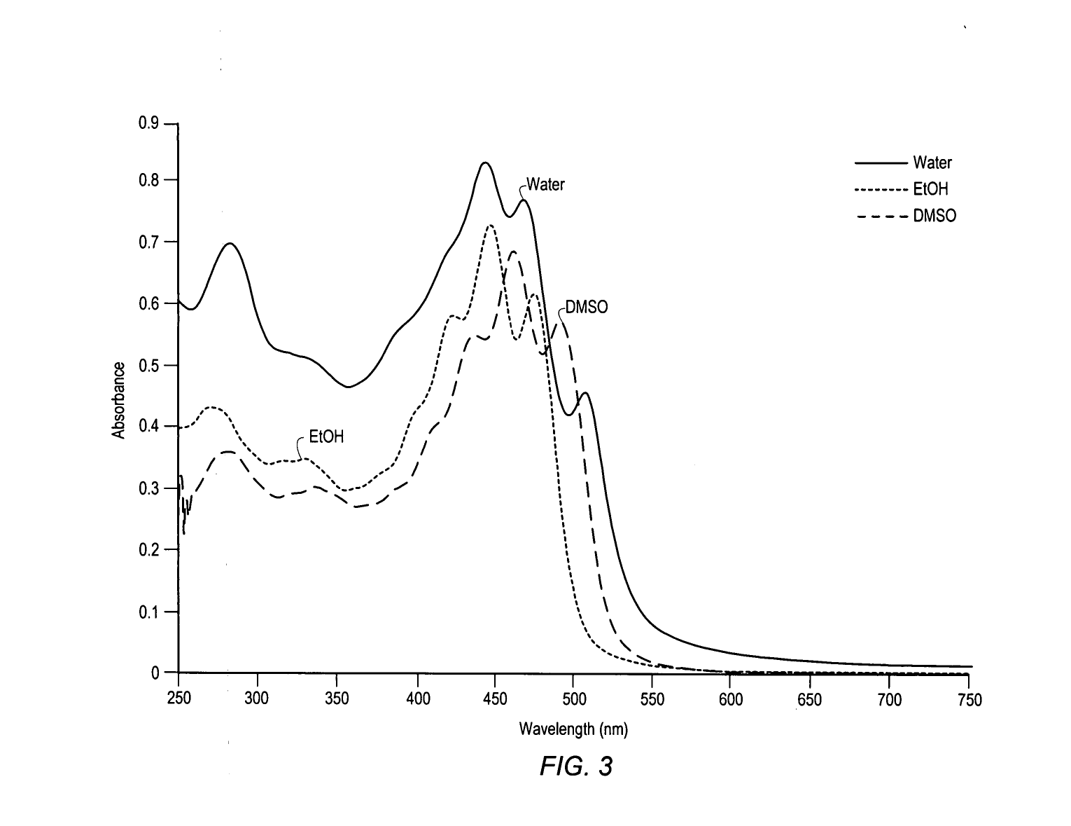

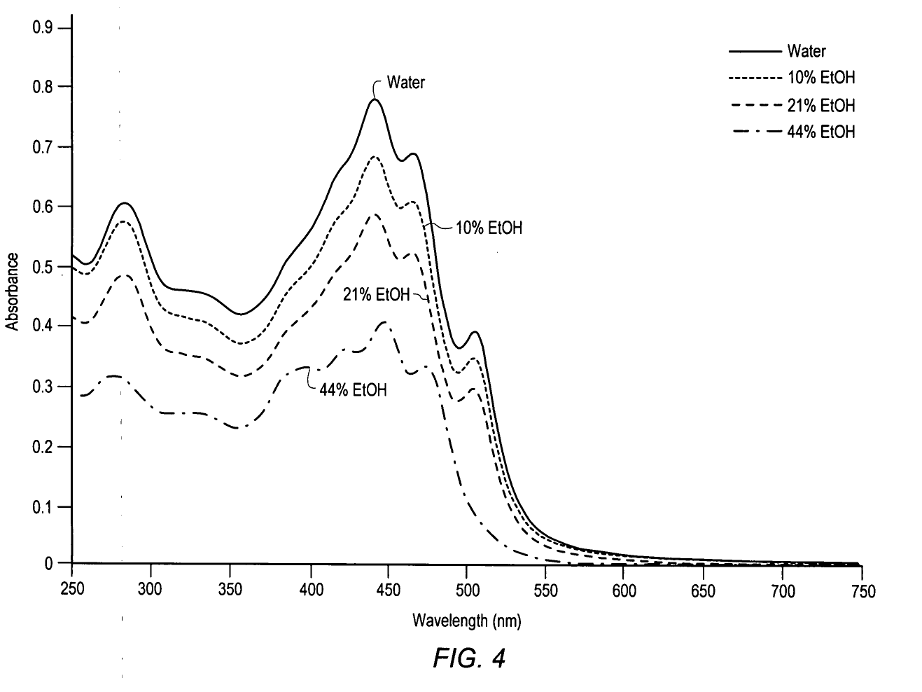

UV-Vis spectral evaluation of the disuccinate lutein sodium salt is depicted in FIG. 2-FIG. 4. FIG. 2 depicts a time series of the UV/Vis absorption spectra of the disodium disuccinate derivative of natural source lutein in water. The λₘₐₓ (443 nm) obtained at time zero did not appreciably blue-shift over the course of 24 hours, vibrational fine structure was maintained (%III/II=35%), and the spectra became only slightly hypochromic (i.e. decreased in absorbance intensity) over time, indicating minimal time-dependent supramolecular assembly (aggregation) of the card-pack type during this time period. Existence of head-to-tail (J-type) aggregation in solution cannot be ruled out. The time series obtained in USP-purified water demonstrated that limited time-dependent aggregation of the card-pack type was observed for this derivative (FIG. 2). Vibrational fine structure was preserved, and a λₘₐₓ of 443 nm (close to the λₘₐₓ for pure lutein in organic solvent) was maintained over the course of 24 hours, which suggested that either supramolecular assembly did not occur in aqueous formulation, or that it was of the “head-to-tail” or J type. FIG. 3 depicts a UV/Vis absorption spectra of the disodium disuccinate derivative of natural source lutein in water (λₘₐₓ=443 nm), ethanol (λₘₐₓ=446 nm), and DMSO (λₘₐₓ=461 nm). Spectra were obtained at time zero. A prominent cis peak is seen with a maximum at 282 nm in water. The expected bathochromic shift of the spectrum in the more polarizable solvent (DMSO) is seen (461 nm). Only a slight hypsochromic shift is seen between the spectrum in water and that in ethanol, reflecting minimal card-pack aggregation in aqueous solution. Replacement of the main visible absorption band observed in EtOH by an intense peak in the near UV region—narrow and displaying no vibrational fine structure—is not observed in the aqueous solution of this highly water-dispersible derivative, in comparison to the spectrum of pure lutein in an organic/water mixture. In organic solvent (EtOH; FIG. 3), a slight red-shift was observed (λₘₐₓ to 446 nm); a bathochromic shift of 18 nm was observed in DMSO, the more polarizable solvent, as expected. Replacement of the main visible absorption band observed in EtOH by an intense peak in the near UV region—narrow and without vibrational fine structure was not observed in the aqueous solution of this highly dispersible derivative. This is in contrast to the behavior of lutein in aqueous formulation. FIG. 4 depicts a UV/Vis absorption spectra of the disodium disuccinate derivative of natural source lutein in water (λₘₐₓ=442 nm) with increasing concentrations of ethanol. The λₘₐₓ increases to 446 nm at an EtOH concentration of 44%, at which point no further shift of the absorption maximum occurs (i.e. a molecular solution has been achieved). Titration with increasing amounts of EtOH (FIG. 4) demonstrated that 44% EtOH was sufficient to shift the λₘₐₓ to 446 nm, identical to that obtained in 100% EtOH (FIG. 3).

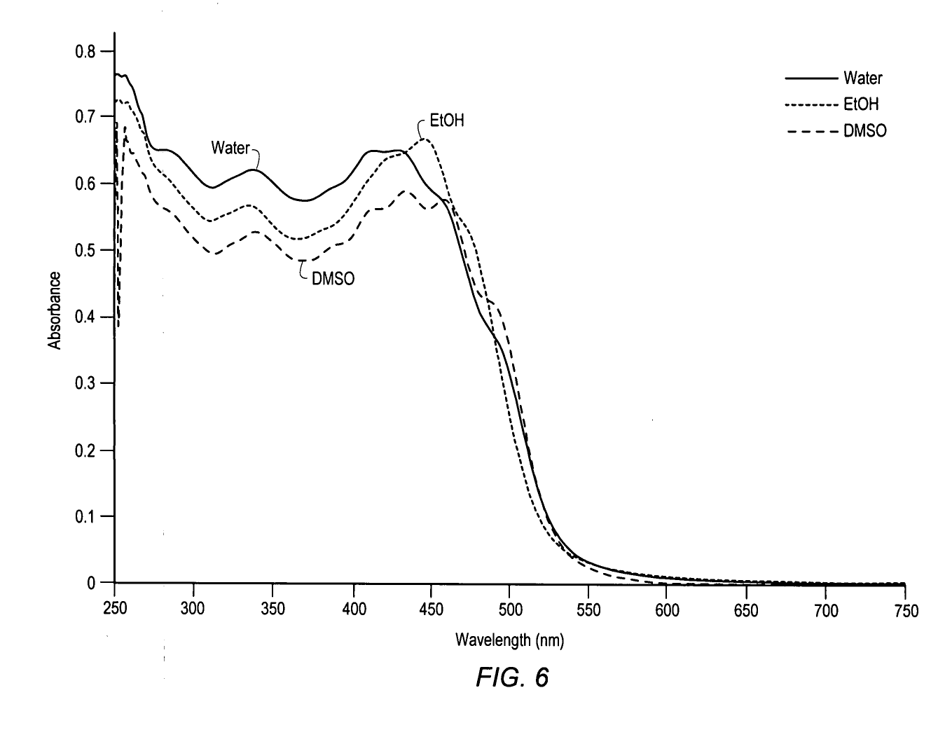

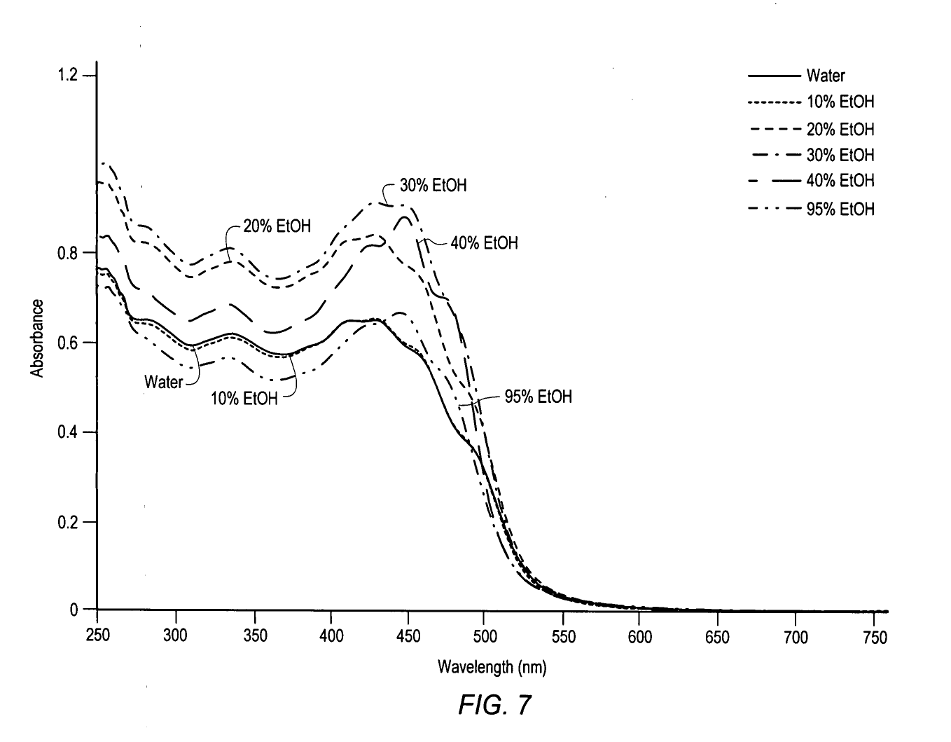

UV-Vis spectral evaluation of the diphosphate lutein sodium salt is depicted in FIG. 5-FIG. 7. FIG. 5 depicts a time series of the UV/Vis absorption spectra of the disodium diphosphate derivative of natural source lutein in water. Loss of vibrational fine structure (spectral distribution beginning to approach unimodality) and the blue-shifted lambda max relative to the lutein chromophore in EtOH suggested that card-pack aggregation was present immediately upon solvation. The λₘₐₓ (428 nm) obtained at time zero did not appreciably blue-shift over the course of 24 hours, and the spectra became slightly more hypochromic over time (i.e. decreased in absorbance intensity), indicating additional time-dependent supramolecular assembly (aggregation) of the card-pack type during this time period. The time series obtained in USP-purified water demonstrated that aggregation of the card-pack (H-type) occurred immediately upon solvation (time zero; FIG. 5), and that additional time-dependent aggregation of the card-pack type was present, but limited for this derivative (FIG. 5). Decreased vibrational fine structure was observed, with the spectrum approaching unimodality with a λₘₐₓ of 428 nm; this spectrum was essentially maintained over the course of 24 hours (compare with FIG. 2, disuccinate lutein sodium salt). FIG. 6 depicts a UV/Vis absorption spectra of the disodium diphosphate derivative of natural source lutein in 95% ethanol (λₘₐₓ=446 nm), 95% DMSO (λₘₐₓ=459 nm), and water (λₘₐₓ=428 nm). Wetting of the diphosphate lutein derivative with a small amount of water was required to obtain appreciable solubility in organic solvent. Spectra were obtained at time zero. The expected bathochromic shift of the spectrum in the more polarizable solvent (95% DMSO) is seen. Increased vibrational fine structure and red-shifting of the spectra were observed in the organic solvents. In organic solvent (95% EtOH; FIG. 6), a red-shift was observed (λₘₐₓ to 446 nm), as was observed with the disuccinate derivate. Wetting the sample with a small amount of water was necessary to achieve suspensions in both EtOH and DMSO. Likewise, the expected bathochromic shift (in this case to 459 nm) in 95% DMSO was also observed for the dipshosphate derivative. FIG. 7 depicts a UV/Vis absorption spectra of the disodium diphosphate derivative of natural source lutein in water (λₘₐₓ=428 nm) with increasing concentrations of ethanol. Concentration of the derivative was held constant for each increased concentration of EtOH in solution. The λₘₐₓ increases to 448 nm at an EtOH concentration of 40%, at which no further shift of the absorption maximum occurs (i.e. a molecular solution is reached). Titration with increasing amounts of EtOH (holding derivative concentration constant) demonstrated that 40% EtOH was sufficient to shift the λₘₐₓ to 446 nm, identical to that obtained in 95% EtOH (FIG. 7).

Direct Superoxide Anion Scavenging by EPR Spectroscopy.

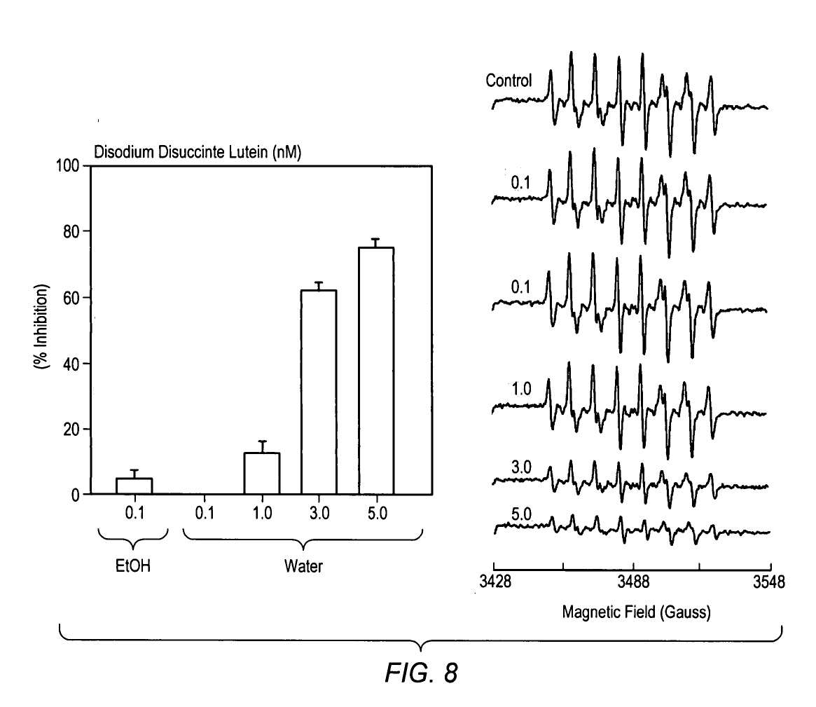

The mean percent inhibition of superoxide anion signal (±SEM) as detected by DEPMPO spin-trap by the disodium disuccinate derivative of natural source lutein (tested in water) is shown in FIG. 8. A 100 μM formulation (0.1 mM) was also tested in 40% EtOH, a concentration shown to produce a molecular (i.e. non-aggregated) solution. As the concentration of the derivative increased, inhibition of superoxide anion signal increased in a dose-dependent manner. At 5 mM, approximately ¾ (75%) of the superoxide anion signal was inhibited. No significant scavenging (0% inhibition) was observed at 0.1 mM in water. Addition of 40% EtOH to the derivative solution at 0.1 mM did not significantly increase scavenging over that provided by the EtOH vehicle alone (5% inhibition). The millimolar concentration scavenging by the derivative was accomplished in water alone, without the addition of organic co-solvent (e.g. acetone, EtOH), heat, detergents, or other additives. This data suggested that card-pack aggregation for this derivative was not occurring in aqueous solution (and thus limiting the interaction of the aggregated carotenoid derivative with aqueous superoxide anion).

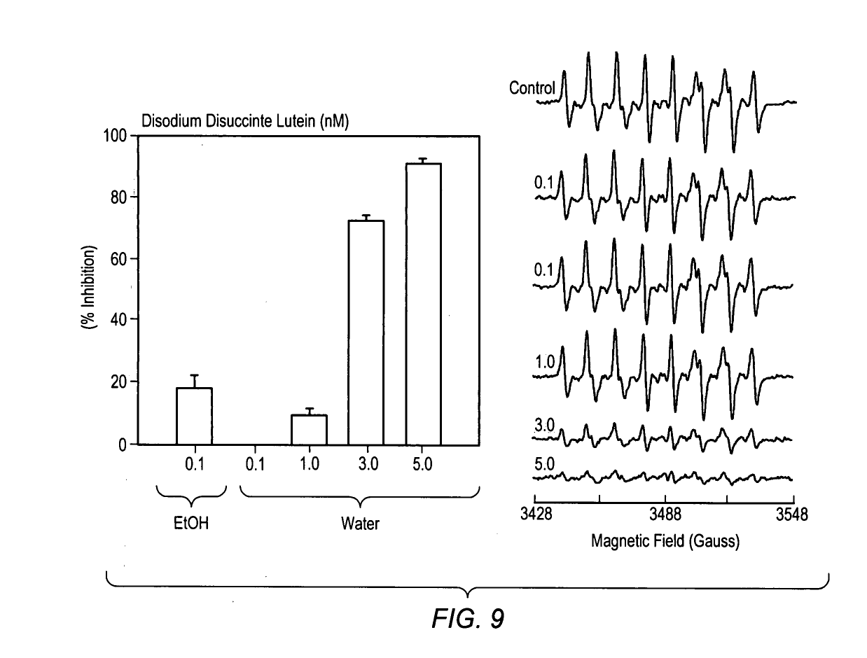

The mean percent inhibition of superoxide anion signal (±SEM) as detected by DEPMPO spin-trap by the disodium diphosphate derivative of natural source lutein (tested in water) is shown in FIG. 9. A 100 μM formulation (0.1 mM) was also tested in 40% EtOH, a concentration also shown to produce a molecular (i.e. non-aggregated) solution of this derivative. As the concentration of the derivative increased, inhibition of the superoxide anion signal increased in a dose-dependent manner. At 5 mM, slightly more than 90% of the superoxide anion signal was inhibited (versus 75% for the disuccinate lutein sodium salt). As for the disuccinate lutein sodium salt, no apparent scavenging (0% inhibition) was observed at 0.1 mM in water. However, a significant increase over background scavenging by the EtOH vehicle (5%) was observed after the addition of 40% EtOH, resulting in a mean 18% inhibition of superoxide anion signal. This suggested that disaggregation of the compound lead to an increase in scavenging ability by this derivative, pointing to slightly increased scavenging ability of molecular solutions of the more water-dispersible diphosphate derivative relative to the disuccinate derivative. Again, the millimolar concentration scavenging by the derivative was accomplished in water alone, without the addition of organic co-solvent (e.g. acetone, EtOH), heat, detergents, or other additives.

TABLE 1
Descriptive statistics of mean % inhibition of superoxide anion signal for aqueous and
ethanolic (40%) formulations of disodium disuccinate derivatives of natural source
lutein tested in the current study. Sample sizes of 3 were evaluated for each formulation,
with the exception of natural source lutein in 40% EtOH stock solution (N = 1).
Mean % inhibition did not increase over background levels until sample concentration
reached 1 mM in water; likewise, addition of 40% EtOH at the 0.1 mM concentration
did not increase scavenging over background levels attributable to the EtOH vehicle
(mean = 5% inhibition).

| | | | | Mean (% | | | | | |
|---|---|---|---|---|---|---|---|---|---|
| Sample | Solvent | Concentration | N | inhibition) | S. D. | SEM | Min | Max | Range |
| Lutein | 40% | 0.1 mM | 3 | 5.0 | 4.4 | 2.5 | 0 | 8 | 8 |
| Disuccinate | EtOH | | | | | | | | |
| Sodium Salt | | | | | | | | | |
| Lutein | Water | 0.1 mM | 1 | 0.0 | ND | ND | 0 | 0 | 0 |
| Disuccinate | | | | | | | | | |
| Sodium Salt | | | | | | | | | |
| Lutein | Water | 1.0 mM | 3 | 13.0 | 5.6 | 3.2 | 8 | 19 | 11 |
| Disuccinate | | | | | | | | | |
| Sodium Salt | | | | | | | | | |
| Lutein | Water | 3.0 mM | 3 | 61.7 | 4.0 | 2.3 | 58 | 66 | 8 |
| Disuccinate | | | | | | | | | |
| Sodium Salt | | | | | | | | | |
| Lutein | Water | 5.0 mM | 3 | 74.7 | 4.5 | 2.6 | 70 | 79 | 9 |
| Disuccinate | | | | | | | | | |
| Sodium Salt | | | | | | | | | |

TABLE 2
Descriptive statistics of mean % inhibition of superoxide anion signal for aqueous and
ethanolic (40%) formulations of disodium diphosphate derivatives of natural source
lutein tested in the current study. Sample sizes of 3 were evaluated for each formulation,
with the exception of lutein diphosphate in water at 100 μM (0.1 mM) where N = 1.
Mean % inhibition of superoxide anion signal increased in a dose-dependent manner
as the concentration of lutein diphosphate was increased in the test assay. At 100 μM
in water, no inhibition of scavenging was seen. The molecular solution in 40% EtOH
(mean % inhibition = 18%) was increased above background scavenging (5%)
by the ethanolic vehicle, suggesting that disaggregation increased scavenging at that
concentration. Slightly increased scavenging (on a molar basis) may have been
obtained with the diphosphate derivative in comparison to disuccinate derivative
(see Table 1 and FIG. 8).

| | | | | Mean (% | | | | | |
|---|---|---|---|---|---|---|---|---|---|
| Sample | Solvent | Concentration | N | inhibition) | S. D. | SEM | Min | Max | Range |
| Lutein | 40% | 0.1 mM | 3 | 18.0 | 7.0 | 4.0 | 11 | 25 | 14 |
| Diphosphate | EtOH | | | | | | | | |
| Sodium Salt | | | | | | | | | |
| Lutein | Water | 0.1 mM | 1 | 0.0 | ND | ND | 0 | 0 | 0 |
| Diphosphate | | | | | | | | | |
| Sodium Salt | | | | | | | | | |
| Lutein | Water | 1.0 mM | 3 | 9.3 | 3.5 | 2.0 | 6 | 13 | 7 |
| Diphosphate | | | | | | | | | |
| Sodium Salt | | | | | | | | | |
| Lutein | Water | 3.0 mM | 3 | 72.3 | 3.1 | 1.8 | 69 | 75 | 6 |
| Diphosphate | | | | | | | | | |
| Sodium Salt | | | | | | | | | |
| Lutein | Water | 5.0 mM | 3 | 91.0 | 2.6 | 1.5 | 88 | 93 | 5 |
| Diphosphate | | | | | | | | | |
| Sodium Salt | | | | | | | | | |

In the current study, facile preparations of the disodium disuccinate and tetrasodium phosphate esters of natural source (RRR) lutein are described. These asymmetric C40 carotenoid derivatives exhibited aqueous dispersibility of 2.85 and 29.27 mg/mL, respectively. Evidence for both card-pack (H-type) and head-to-tail (J-type) supramolecular assembly was obtained with UV-Vis spectroscopy for the aqueous solutions of these compounds. Electronic paramagnetic spectroscopy of direct aqueous superoxide scavenging by these derivatives demonstrated nearly identical dose-dependent scavenging profiles, with slightly increased scavenging noted for the diphosphate derivative. In each case, scavenging in the millimolar range was observed. These results suggest that as parenteral soft drugs with aqueous radical scavenging activity, both compounds are potentially useful in those clinical applications in which rapid and/or intravenous delivery is desired for the desired therapeutic effect(s). These compounds can also be used to overcome problems with oral bioavailability in mammals, due to their facile parenteral administration as aqueous formulations. Nadolski et al. 2006 describes methods of synthesis and testing of these compounds, and is incorporated by reference as though fully set forth herein.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of treating a visual disability comprising administering to a subject a therapeutically effective amount of a pharmaceutically acceptable composition comprising one or more carotenoid derivatives or analogs having the structure: 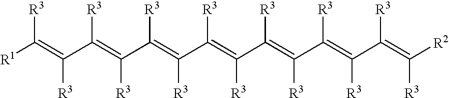
where each R³ is independently hydrogen or methyl, and where each R¹ and R² are independently: 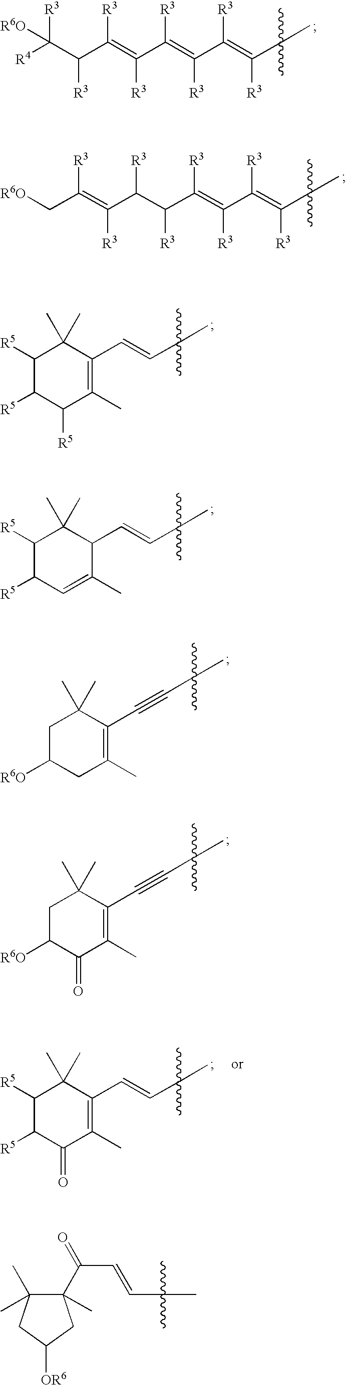
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; —aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

2. The method of claim 1, further comprising repigmenting a macula.

3. The method of claim 1, further comprising stabilizing visual acuity.

4. The method of claim 1, further comprising improving visual acuity.

5. The method of claim 1, wherein treating a visual disability further comprises treating macular degeneration.

6. The method of claim 5, wherein macular degeneration comprises age-related macular degeneration.

7. The method of claim 5, wherein macular degeneration comprises “wet” macular degeneration.

8. The method of claim 5, wherein macular degeneration comprises “dry” macular degeneration.

9. (canceled)

10. (canceled)

11. The method of claim 1, wherein the composition comprises one or more carotenoid derivatives or analogs having the structure: 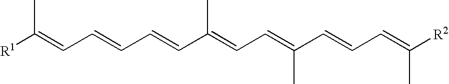
where each R¹ and R² are independently: 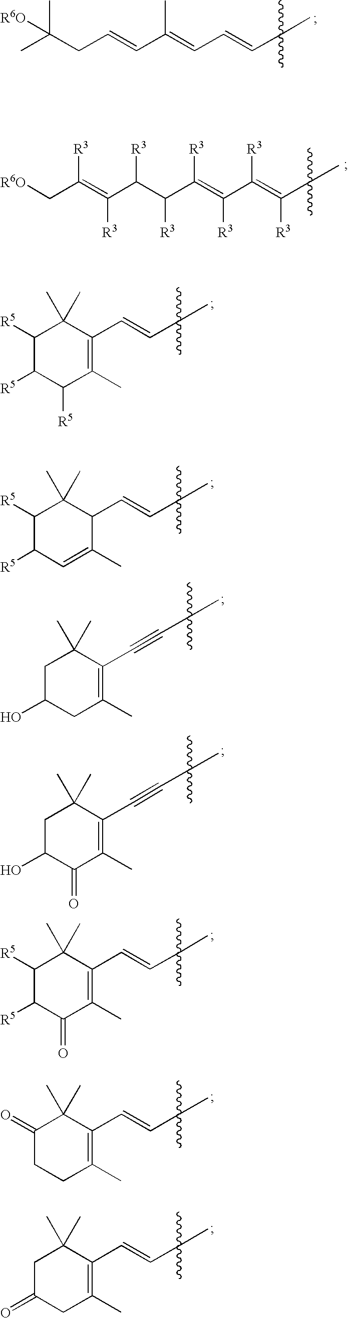 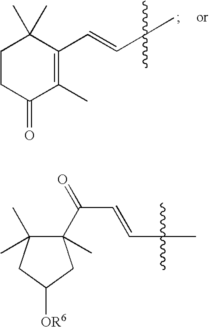
where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂—; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

12. The method of claim 1, wherein the composition comprises one or more carotenoid derivatives or analogs having the structure: 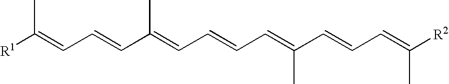
where each R¹ and R² are independently: 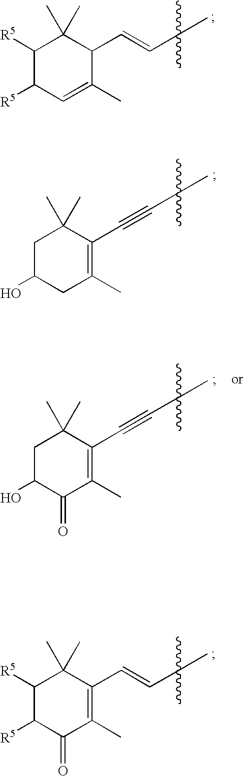
where each R⁵ is independently hydrogen, —OH, or —OR⁶ wherein at least one R⁵ group is —OR⁶; wherein each R⁶ is independently: 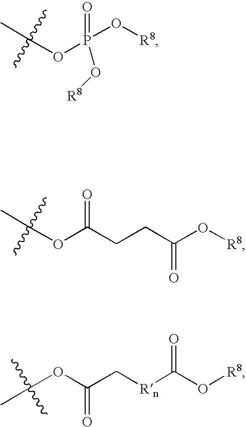
or a co-antioxidant; wherein R⁸ is hydrogen, alkyl, aryl, benzyl, Group IA metal, or a co-antioxidant; wherein R′ is CH₂; and where n is 1 to 9.

13. The method of claim 1, wherein the composition comprises one or more carotenoid derivatives or analogs having the structure: 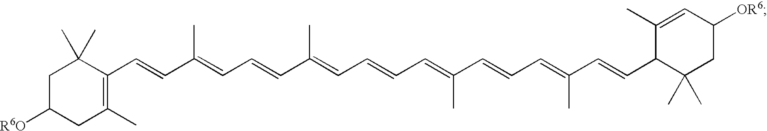
wherein each —OR⁶ is independently: 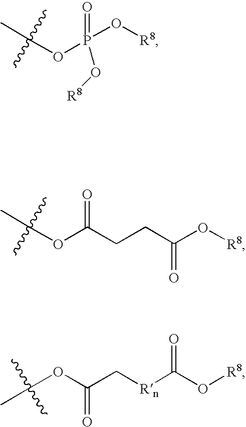
or a co-antioxidant; wherein R⁸ is hydrogen, alkyl, aryl, benzyl, Group IA metal, or a co-antioxidant; wherein R′ is CH₂; and where n is 1 to 9.

14. The method of claim 1, wherein the composition comprises one or more carotenoid derivatives or analogs having the structure: 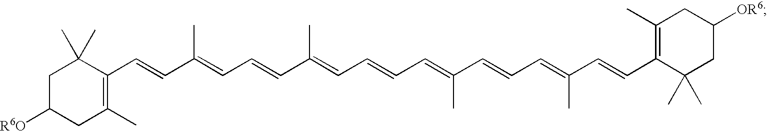
wherein each —OR is independently: 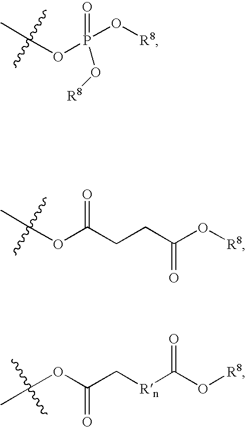
or a co-antioxidant; wherein R⁸ is hydrogen, alkyl, aryl, benzyl, Group IA metal, or a co-antioxidant; wherein R′ is CH₂; and where n is 1 to 9.

15. The method of claim 1, wherein the composition comprises two or more carotenoid derivatives or analogs having the structures: 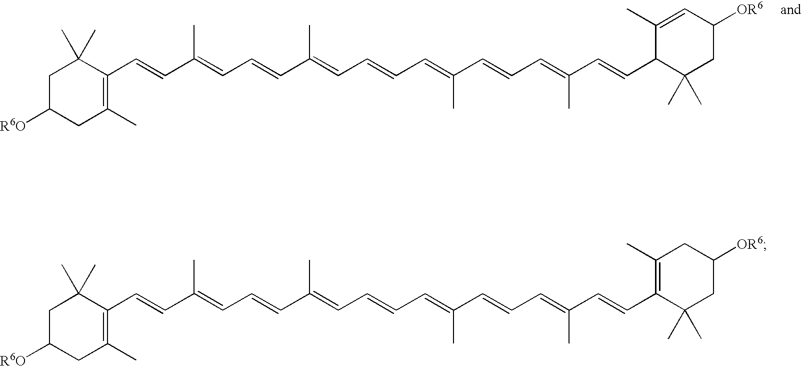
wherein each —OR⁶ is independently: 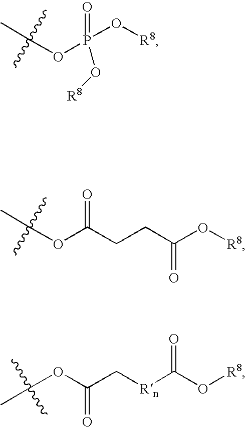
or a co-antioxidant; wherein R⁸ is hydrogen, alkyl, aryl, benzyl, Group IA metal, or a co-antioxidant; wherein R′ is CH₂; and where n is 1 to 9.

16. The method of claim 1, wherein each —OR⁶ independently comprises: 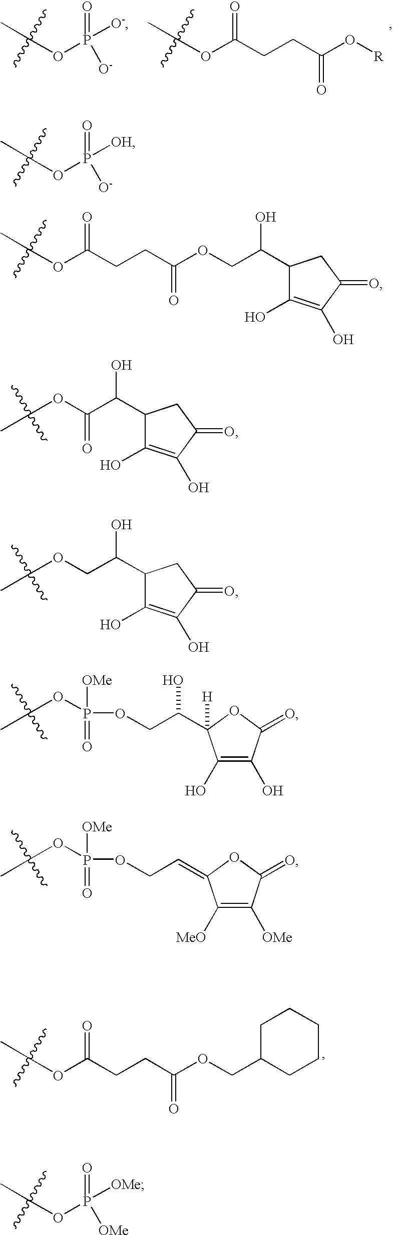
and wherein each R is independently H, alkyl, aryl, benzyl, Group IA metal, or co-antioxidant.

17. The method of claim 1, wherein each —OR⁶ independently comprises: 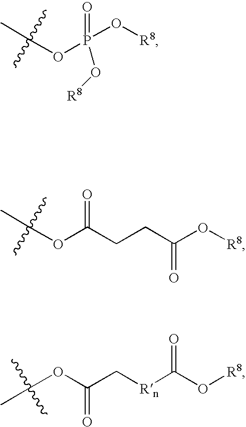
or a co-antioxidant; wherein R⁸ is hydrogen, alkyl, aryl, benzyl, Group IA metal, or a co-antioxidant; wherein R′ is CH₂; and where n is 1 to 9.

18. (canceled)

19. (canceled)

20. (canceled)

21. (canceled)

22. The method of claim 1, wherein the composition comprises one or more carotenoid derivatives or analogs having the structures: 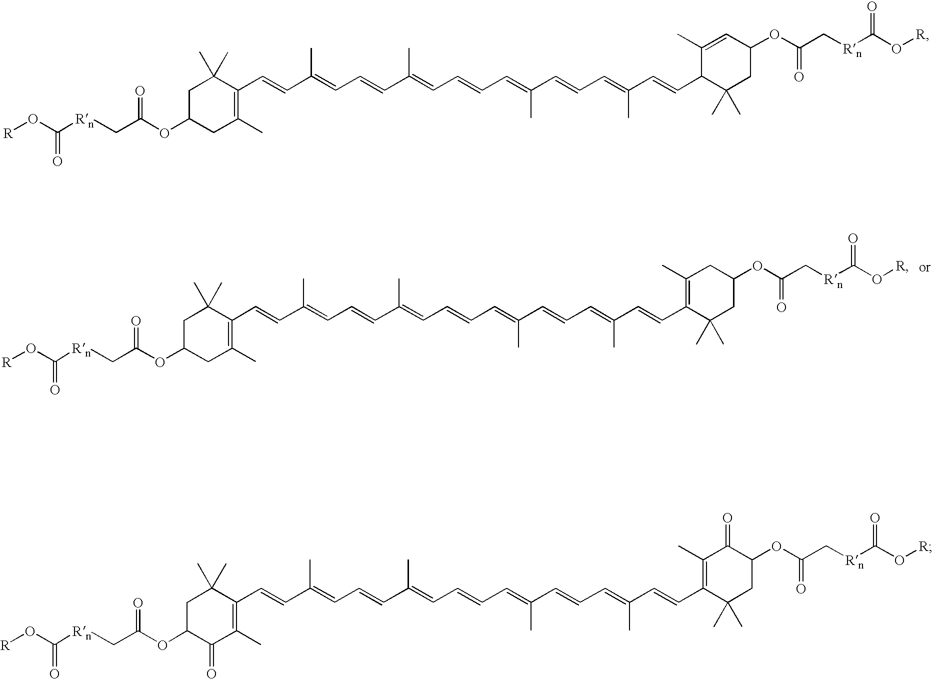
where each R is independently H, alkyl, aryl, benzyl, Group IA metal, or a co-antioxidant.

23. The method of claim 1, wherein the composition comprises one or more carotenoid derivatives or analogs having the structures: 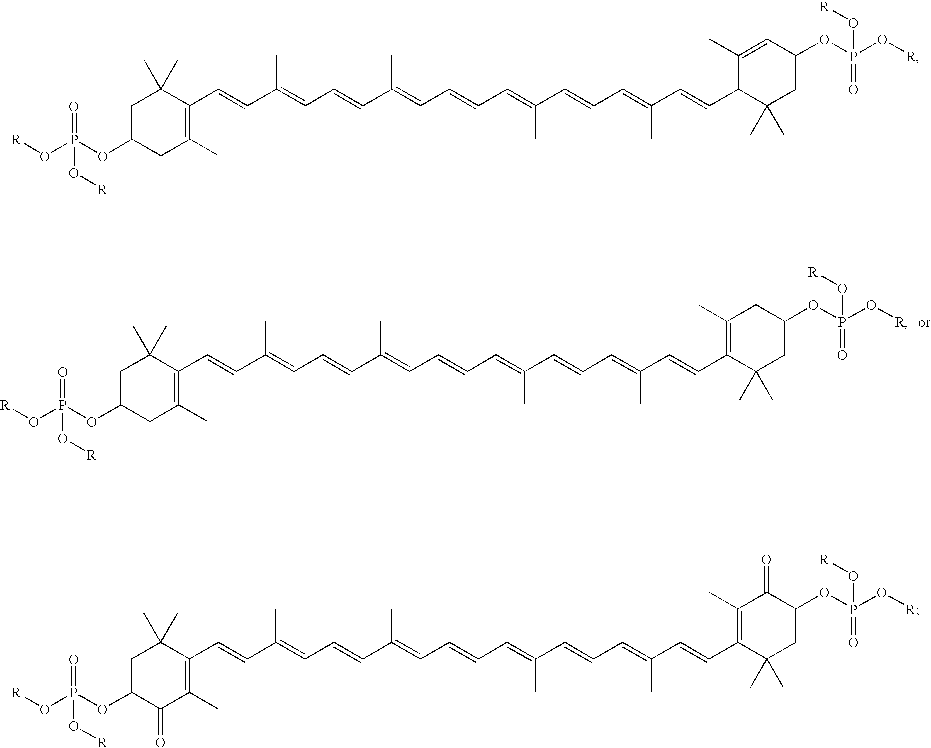
where each R is independently H, alkyl, aryl, benzyl, Group IA metal, or a co-antioxidant.

24. The method of claim 1, wherein the co-antioxidant comprises Vitamin C, Vitamin C analogs, Vitamin C derivatives, Vitamin E, Vitamin E analogs, Vitamin E derivatives, flavonoids, flavonoid derivatives, or flavonoid analogs.

25. The method of claim 24, wherein the flavonoids comprise quercetin, xanthohumol, isoxanthohumol, or genistein.

26. The method of claim 1, wherein the composition comprises one or more carotenoid derivatives or analogs having the structures: 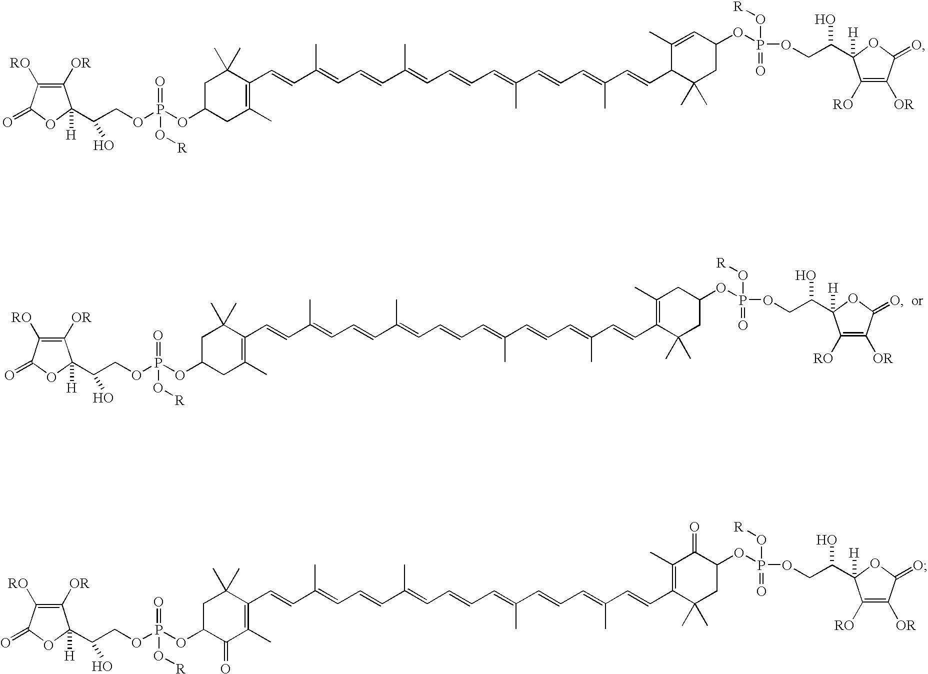
where each R is independently H, alkyl, aryl, benzyl, or a Group IA metal.

27-153. (canceled)
